(19) United States
(12) Patent Application Publication
(10) Pub. No.: US 2015/0218227 A1
(43) Pub. Date: Aug. 6, 2015

(54) PEPTIDES FOR THE BINDING OF NUCLEOTIDE TARGETS

(71) Applicants: THE UNIVERSITY OF WESTREN AUSTRALIA, Crawley, Nedland (AU); THE STATE BOARD OF HIGHER EDUCATION ON BEHALF OF THE UNIVERSITY OF OREGON, Eugene, OR (US)

(72) Inventors: Alice Barkan, Eugene, OR (US); Ian Small, Wattle Grove (AU); Margarita Rojas, Eugene, OR (US); Charles Bond, Wembley (AU); Sota Fujii, Soraku-gun (JP); Yee Seng Chong, West Perth (AU)

(21) Appl. No.: 14/394,945

(22) PCT Filed: Apr. 16, 2013
(86) PCT No.: PCT/AU2013/000387

§ 371 Date: Oct. 16, 2014

(30) Foreign Application Priority Data
Apr. 16, 2012 (AU) 2012901486
Jul. 10, 2012 (AU) 2012902961

(51) Int. Cl. C07K 14/00; C12N 15/10

(52) U.S. Cl. CPC C07K 14/001; C12N 15/1062

(57) ABSTRACT

A method of regulating expression of a gene in a cell is described, comprising the step of introducing into the cell a recombinant polypeptide comprising a PPR RNA-binding domain which itself comprises at least a pair of PPR RNA base-binding motifs. The PPR RNA base-binding motifs of the PPR RNA-binding domain are operably capable of binding the target RNA molecule with a target RNA sequence. Recombinant polypeptides comprising at least one PPR RNA-binding domain capable of binding to target RNA sequence are also described, together with fusion proteins comprising the recombinant PPR RNA-binding domains as well as isolated nucleic acids useful in preparing the recombinant polypeptides described. Recombinant vectors; compositions comprising the recombinant polypeptides; isolated nucleic acids; recombinant vectors; host cells comprising same; use of same in the manufacture of a medicament for regulating gene expression; as well as systems and kits for regulating gene expression are also described.

### ACKNOWLEDGMENT OF GOVERNMENT SUPPORT

This invention was made in part with government support under grant number MCB-0940979 awarded by the National Science Foundation. The United States Government has certain rights in the invention.

### TECHNICAL FIELD

The invention relates to methods of regulating the expression of a gene in a cell; methods of identifying a binding target RNA sequence of a PPR RNA-binding domain; as well as recombinant polypeptides; fusion proteins comprising the recombinant polypeptides; isolated nucleic acids; recombinant vectors; compositions comprising the recombinant polypeptides, nucleic acids, or recombinant vectors of the invention; use of same in the manufacture of the medicament for regulating gene expression; systems and kits for regulating gene expression, and host cells.

### BACKGROUND ART

Gene expression and protein production in cells is regulated in many ways, including regulating the extent of chromatin structure, epigenetic control, transcriptional initiation and control of the rate thereof, messenger RNA (mRNA) transcript processing and modification, mRNA transport, mRNA transcript stability, translational initiation, control of transcript levels by small non-coding RNAs, post-translational modification, protein transport, and control of protein stability.

The ability to specifically regulate gene expression has broad application in various fields including biochemistry, molecular biology, biotechnology, and pharmaceutics. Attempts to recombinantly regulate gene expression have involved many different kinds of approaches including those of RNA interference (RNAi) technologies, antisense RNA (aRNA) technologies, and more recently the recombinant engineering of RNA binding proteins such as PUF proteins.

While RNAi and aRNA are well-established technologies for gene expression regulation by specific targeting of mRNA transcripts, the design and production of effective RNA molecules can be both challenging and complex. Disadvantages of RNAi can include non-specific binding, the need for transfection reagents or delivery vehicles, low and variable transfection efficiency, partial and transient gene suppression effects, dependence upon processing by RNAi machinery, and undesirable immunogenic effects.

RNA binding proteins, such as PUF (Drosophila Pumilio (Pum) and C. elegans FBF (fem-3 binding factor)) proteins, have more recently been proposed as alternatives for use in regulating gene expression. RNA binding proteins are often more stable than RNAi and aRNA molecules. However, most known RNA binding proteins are poor candidates for engineering due to the difficulty of predicting their sequence specificities.

PUF proteins have been suggested for use in the engineering of proteins with specified sequence preferences. PUF domains consist of eight triple-helix bundles that stack, to form a crescent shaped solenoid and regulate the expression of specific sets of cytosolic mRNAs in eucaryotes. Crystal structures of PUF-RNA complexes revealed a mechanism for RNA recognition, in which several amino acids in each repeat recognize a single RNA base which specify the binding of individual PUF repeats to specific nucleotides. However, the recombinant engineering of PUF proteins for applications in the regulation of gene expression is limited. PUF proteins demonstrate low genetic diversity, implying substantial constraints on their repertoire of potential ligands. PUF domains consist of 8 repeats and bind sites of 8-9 nucleotides that share sequence similarity. This relatively small natural diversity suggests that the functional potential of PUF domains for targeted binding of desired RNA sequences may be limited.

Pentatricopeptide repeat (PPR) proteins, a family of RNA binding proteins belonging to the alpha solenoid repeat superfamily, have been suggested for use in engineering of RNA binding proteins for the preferential binding of specific RNA sequences. PPR proteins typically bind single-stranded RNA in a sequence-specific fashion. However, the basis for sequence-specific RNA recognition by PPR tracts is unknown. PPR proteins are found in eucaryotes. The PPR family in the plant lineage is notable for its size, with ˜450 members in angiosperms, where they localise primarily to mitochondria and chloroplasts and influence various aspects of RNA metabolism. Many PPR proteins are essential for photosynthesis or respiration, and PPR-encoding genes are associated with genetic diseases in humans, suggesting that not all naturally occurring mutations in PPR-encoding genes are tolerated.

PPR proteins harbor short helical repeats that stack to form surfaces suited for the binding of macromolecules. PPR proteins are defined by tandem arrays of degenerate 35 amino acid repeats, which fold into 2-helix bundles that stack to form domains having broad RNA-binding surfaces, the structural detail of which is as yet unclear. PPR domains are variable in length, having between 2 and 30 repeats, and average ˜12 repeats. PPR proteins fall into several subfamilies, including “P-type” PPR proteins and “PLS” PPR proteins, that differ in repeat organization and in the presence of accessory domains. P-type PPR proteins influence organellar RNA splicing, stabilization, translation, and processing, whereas PLS proteins function primarily in RNA editing. P-type PPR tracts bind only to single-stranded RNA. Organellar RNA editing factors are from the “PLS” subfamily, which is characterized by alternating canonical, “long”, and “short” PPR motifs.

While PPR proteins have been attributed to RNA binding functions in general, the specific nature and mechanism of this binding has remained unclear. PPR proteins have diverse RNA ligands and functions. Only about 50 PPR proteins have been assigned a general RNA binding function based on molecular defects in loss-of-function mutants. Typically, PPR proteins are required for post-transcriptional steps in organellar gene expression (e.g. RNA splicing, editing, stabilization, and translation) and are therefore believed to be required for photosynthesis or respiration. The understanding of PPR protein function between species has been complicated by the evolutionary fluidity of PPR-RNA interactions. Specific functions have been assigned to only a small fraction of the ˜450 PPR proteins in crop and model angiosperms.

In light of limited information on PPR function, it is not currently possible to design PPR proteins to bind arbitrary RNA sequences, as has been proposed with other proteins, namely PUF domain proteins. The minimal combination of residues required to specify the nucleotide ligands of PPR motifs are unclear. This information is essential for the design of any recombinant PPR proteins intended to specifically bind target RNA sequences.

Most protein-nucleic acid interactions are idiosyncratic, and lack the predictability necessary to engineer specific interactions.

There thus exists a continued need for alternative methods for the specific regulation of gene expression and for agents for use therein. The present invention seeks to ameliorate, one or more of the deficiencies of the prior art mentioned above.

The above discussion of the background art is intended to facilitate an understanding of the present invention only. The discussion is not an acknowledgement or admission that any of the material referred to is or was part of the common general knowledge as at the priority date of the application.

### SUMMARY OF INVENTION

According to the invention there is provided a recombinant polypeptide comprising at least one PPR RNA-binding domain capable of binding to a target RNA sequence, the PPR RNA-binding domain comprising at least two PPR RNA base-binding motifs selected from the group comprising:

a.
i. amino acid position six of a first PPR RNA base-binding motif selected from the group comprising threonine (T), serine (S), and glycine (G);
ii. amino acid position one of a second adjacent PPR binding motif selected from the group comprising asparagine (N), threonine (T), and serine (S); and
iii. the PPR domain is operably capable of binding to an adenine (A) RNA base in a target RNA sequence;

b.
i. amino acid position six of the first PPR RNA base-binding motif is selected from the group comprising threonine (T), serine (S), glycine (G), and alanine (A);
ii. amino acid position one of the second adjacent PPR binding motif is selected from the group comprising aspartic acid (D), threonine (T), and serine (S); and
iii. the PPR domain is operably capable of binding to a guanine (G) RNA base in a target RNA sequence;

c.
i. amino acid position six of the first PPR RNA base-binding motif is threonine (T) or asparagine (N);
ii. amino acid position one of the second adjacent PPR binding motif is selected from the group comprising asparagine (N), serine (S), aspartic acid (D), and threonine (T); and
iii. the PPR domain is operably capable of binding to a cytosine (C) RNA base in a target RNA sequence; and

d.
i. amino acid position six of the first PPR RNA base-binding motif is threonine (T) or asparagine (N);
ii. amino acid position one of the second adjacent PPR binding motif is selected from the group comprising aspartic acid (D), serine (S), asparagine (N), and threonine (T); and
iii. the PPR domain is operably capable of binding to a uracil (U) RNA base in a target RNA sequence.

In a preferred embodiment of the invention, amino acid position six of the first PPR RNA base-binding motif is asparagine (N), amino acid position one of the second adjacent PPR binding motif is serine (S), and the PPR domain is operably capable of binding to a cytosine (C) RNA base in a target RNA sequence.

In another preferred embodiment of the invention, amino acid position six of the first PPR RNA base-binding motif is asparagine (N), amino acid position one of the second adjacent PPR binding motif is serine (S), and the PPR domain is operably capable of binding to either a cytosine (C) RNA base or a uracil (U) RNA base in a target RNA sequence.

In another preferred embodiment of the invention, amino acid position six of the first PPR RNA base-binding motif is asparagine (N), amino acid position one of the second adjacent PPR binding motif is aspartic acid (D), and the PPR domain is operably capable of binding to either a cytosine (C) RNA base for a uracil (U) RNA base in a target RNA sequence.

In another preferred embodiment of the invention, amino acid position six of the first PPR RNA base-binding motif is serine (S), amino acid position one of the second adjacent PPR binding motif is aspartic acid (D), and the PPR domain is operably capable of binding to a guanine (G) RNA base in a target RNA sequence.

In another preferred embodiment of the invention, amino acid position six of the first PPR RNA base-binding motif is glycine (G), amino acid position one of the second adjacent PPR binding motif is aspartic acid (D), and the PPR domain is operably capable of binding to a guanine (G) RNA base in a target RNA sequence.

In another preferred embodiment of the invention, amino acid position six of the first PPR RNA base-binding motif is glycine (G), amino acid position one of the second adjacent PPR binding motif is asparagine (N), and the PPR domain is operably capable of binding to an adenine (A) RNA base in a target RNA sequence.

In another preferred embodiment of the invention, amino acid position six of the first PPR RNA base-binding motif is threonine (T), amino acid position one of the second adjacent PPR binding motif is aspartic acid (D), and the PPR domain is operably capable of binding to a guanine (G) RNA base in a target RNA sequence.

In another preferred embodiment of the invention, amino acid position six of the first PPR RNA base-binding motif is threonine (T), amino acid position one of the second adjacent PPR binding motif is asparagine (N), and the PPR domain is operably capable of binding to an adenine (A) RNA base in a target RNA sequence.

In another preferred embodiment of the invention, amino acid position six of the first PPR RNA base-binding motif is asparagine (N), amino acid position one of the second adjacent PPR binding motif is asparagine (N), and the PPR domain is operably capable of binding equally to either a cytosine (C) RNA base or a uracil (U) RNA base in the target RNA sequence.

In another preferred embodiment of the invention, amino acid position six of the first PPR RNA base-binding motif is asparagine (N), amino acid position one of the second adjacent PPR binding motif is serine (S), and the PPR domain is operably capable of binding to either a cytosine (C) RNA base or a uracil (U) RNA base in the target RNA sequence, but with a preference in binding to a cytosine (C) RNA base. That is, cytosine (C) is bound by the PPR domain with higher affinity than uracil (U).

In another preferred embodiment of the invention, amino acid position six of the first PPR RNA base-binding motif is asparagine (N), amino acid position one of the second adjacent PPR binding motif is aspartic acid (D), and the PPR domain is operably capable of binding to a uracil (U) RNA base and to a cytosine (C) RNA base in the target RNA sequence, but with a preference in binding to a uracil (U) RNA base. That is, cytosine (C) is bound by the PPR domain with lower affinity than uracil (U).

In another preferred embodiment of the invention, amino acid position six of the first PPR RNA base-binding motif is threonine (T), amino acid position one of the second adjacent PPR binding motif is threonine (T), and the PPR domain is operably capable of binding to a adenine (A) RNA, to cytosine (C), to uracil (U), and to guanine (G), but with a preference in binding to a adenine (A) RNA base. That is, adenine (A) is bound by the PPR domain with higher affinity than any of cytosine (C), to uracil (U), and to guanine (G). In this embodiment of the invention the PPR domain is operably equally capable of binding to cytosine (C) and to uracil (U). In this embodiment of the invention, the PPR domain is operably capable of binding to guanine (G), but with a lower affinity than to adenine (A), cytosine (C) or uracil (U). That is, the preference in binding affinity of the PPR domain of this embodiment of the invention is as follows: adenine (A)>cytosine (C), uracil (U)>guanine (G).

In another preferred embodiment of the invention, amino acid position six of the first PPR RNA base-binding motif is threonine (T), amino acid position one of the second adjacent PPR binding motif is serine (S), and the PPR domain is operably capable of binding to a adenine (A) RNA, to cytosine (C), to uracil (U), and to guanine (G), but with a preference in binding to a adenine (A) RNA base. That is, adenine (A) is bound by the PPR domain with higher affinity than to any of cytosine (C), uracil (U), or guanine (G). In this embodiment of the invention the PPR domain is operably equally capable of binding to cytosine (C) and to uracil (U). In this embodiment of the invention, the PPR domain is operably capable of binding to guanine (G), but with a lower affinity than to adenine (A), cytosine (C) or uracil (U). That is, the preference in binding affinity of the PPR domain of this embodiment of the invention is as follows: adenine (A)>cytosine (C), uracil (U)>guanine (G).

Binding of the identified amino acids in the PPR domain to the identified RNA nucleotides in the RNA target sequence may be at different affinities.

Further features of the invention provide for each PPR RNA base-binding motif to comprise between 30 and 40 amino acids.

Still further features of the invention provide for the PPR RNA-binding domain to comprise a plurality of pairs of PPR RNA base-binding motifs. Further, the plurality of PPR RNA base-binding motifs may comprise a first pair of PPR RNA base-binding motifs capable of binding to a first RNA base and a second pair of PPR RNA base-binding motifs capable of binding to a second RNA base, wherein the first and second pairs of PPR RNA base-binding motifs enhance the binding of the RNA bases when the RNA bases are provided in the form of single stranded RNA.

In one embodiment of the invention, the PPR RNA-binding domain comprises a plurality of consecutively ordered pairs of PPR RNA base-binding motifs operable to bind a target RNA molecule with a target RNA sequence, each pair of PPR RNA base-binding motifs capable of specifically binding to a cytosine (C), adenine (A), guanine (G), or uracil (U) RNA base in a target RNA sequence, wherein the consecutive order of the pairs of PPR RNA base-binding motifs corresponds with the consecutive order of the target RNA sequence.

The target RNA molecule may be RNA encoding a reporter protein including, but not limited to, his3, β-galatosidase, GFP, RFP, YFP, luciferase, β-glucuronidase, and alkaline phosphatase.

The target RNA molecule may be RNA transcribed from chloroplast and/or mitochondrial genes. The chloroplast and/or mitochondrial genes may be endogenous or exogenous. Furthermore, the target RNA molecule may be derived or expressed by a plant cell, such as, but not limited to, a tobacco plant cell.

The target RNA molecule may be encoded in a transgene that is introduced into a cell such that an endogenous PPR protein will affect the expression of the transgene through the known binding pattern identified herein. The transgene may encode a reporter protein or protein that mediates a desired biological activity (e.g. growth, maturation rate, resistance, etc.)

Further features of the invention provide for the plurality of RNA base-binding motifs to comprise between 2 and 40 PPR RNA base-binding motifs, preferably between 8 and 20 PPR RNA base-binding motifs.

Yet further features provide for the PPR RNA-binding domain to comprise a plurality of pairs of PPR RNA base-binding motifs operably linked via amino acid spacers; for such amino acid spacers to include those typically used by persons skilled in the art; such as, but not limited to, synthetic amino acid spacers, and further for the amino acid spacers to be derived, wholly or in part, from PPR proteins derived from one or more of the group comprising Zea Mays (maize), Oryza sativa (Asian rice), Oryza glaberrima (African rice), Hordeum spp. (Barley), Arabidopsis spp. (Rockcress) such as Arabidopsis thaliana, or any other species harboring PPR proteins.

The above PPR proteins are given as examples and it will be appreciated that these examples are intended for the purpose of exemplification. PPR proteins comprise an extensive family of proteins and the invention may be applied to recombinant proteins derived from a large range of PPR proteins which may be functionally equivalent to those described herein. It is understood that PPR proteins demonstrating amino acid sequence homology or similarity to those described herein may be useful for the present invention. It will be also appreciated that many PPR proteins may not demonstrate amino acid sequence similarity to those described herein, yet may demonstrate secondary and tertiary structural and functional similarity and/or equivalence to other PPR proteins. The present invention is not limited to PPR proteins demonstrating amino acid sequence homology or similarity to those described herein, and includes PPR proteins that demonstrate functional secondary and tertiary structural and/or functional similarity to the embodiments described herein. Examples of such proteins include PPR proteins derived from mammals, including but not limited to human PPR proteins such as LRPPRC (Leucine-rich PPR-motif Containing protein). Further examples of such proteins include PPR proteins derived from pathogens and microorganisms causing disease.

In another preferred embodiment of the invention, the amino acid spacers are derived from SEQ ID NO: 4, or part thereof.

The invention also provides a fusion protein comprising at least one PPR RNA-binding domain capable of specifically binding to an RNA base, and an effector domain.

The invention also provides a fusion protein comprising at least one recombinant polypeptide of the invention, and an effector domain.

The effector domain may be any domain capable of interacting with RNA, whether transiently or irreversibly, directly or indirectly, including but not limited to an effector domain selected from the group comprising; Endonucleases (for example RNase III, the CRR22 DYW domain, and Dicer); proteins and protein domains responsible for stimulating RNA cleavage (for example CPSF, CstF, CFIm and CFIIm); Exonucleases (for example XRN-1, Exonuclease T); Deadenylases (for example HNT3); proteins and protein domains responsible for nonsense mediated RNA decay (for example UPF1, UPF2, UPF3, UPF3b, RNP S1, Y14, DEK, REF2, and SRm160); proteins and protein domains responsible for stabilizing RNA (for example PABP); proteins and protein domains responsible for repressing translation (for example Agog and Ago4); proteins and protein domains responsible for stimulating translation (for example Staufen); proteins and protein domains responsible for polyadenylation of RNA (for example PAP1, GLD-2, and Star-PAP); proteins and protein domains responsible for polyuridinylation of RNA (for example CID1 and terminal uridylate transferase); proteins and protein domains responsible for RNA localization (for example IMP1, ZBP1, She2p, She3p, and Bicaudal-D); proteins and protein domains responsible for nuclear retention of RNA (for example Rrp6); proteins and protein domains responsible for nuclear export of RNA (for example TAP, NXF1, THO, TREX, REF, and Aly); proteins and protein domains responsible for repression of RNA splicing (for example PTB, Sam68, and hnRNP Al); proteins and protein domains responsible for stimulation of RNA splicing (for example Serine/Arginine-rich (SR) domains); proteins and protein domains responsible for reducing the efficiency of transcription (for example FUS (TLS)); proteins and protein domains responsible for stimulating transcription (for example CDK7 and HIV Tat), and deaminases such as the DYW domain, APOBEC, and adenine deaminase.

The effector domain may also be a reporter protein, or functional fragment thereof, including, but not limited to, his3, β-galatosidase, GFP, RFP, YFP, luciferase, β-glucuronidase, and alkaline phosphatase.

The recombinant PPR polypeptide may be derived from a P-type PPR protein, such as, but not limited, to the Rf clade of fertility restorers.

Further features provide for the PPR RNA-binding domain and the effector domain to be operably linked via a peptide spacer.

Due to the degeneracy of the DNA code, it will be well understood to one of ordinary skill in the art that substitution of nucleotides may be made without changing the amino acid sequence of the polypeptide. Therefore, the invention includes any nucleic acid sequence for a recombinant polypeptide comprising a recombinant PPR RNA-binding domain according to the invention capable of specifically binding to an RNA base. Moreover, it is understood in the art that for a given protein's amino acid sequence, substitution of certain amino acids in the sequence can be made without significant effect on the function of the peptide. Such substitutions are known in the art as “conservative substitutions.” The invention encompasses a recombinant polypeptide comprising a PPR RNA-binding domain that contains conservative substitutions, wherein the function of the recombinant polypeptide in the specific binding of an RNA base according to the invention is not altered. Generally, the identity of such a mutant recombinant polypeptide comprising a PPR RNA-binding domain will be at least 40% identical to a polypeptide encoded by the sequence of any one of SEQ ID NOS: 5-21. More preferably, the mutant recombinant polypeptide comprising a PPR RNA-binding domain will be at least 45%; at least 50%; at least 55%; at least 60%; at least 65%; at least 70%; at least 75%; at least 80%; at least 85%; at least 90%; at least 95%; or at least 97% identical; to a polypeptide encoded by the sequence of any one of SEQ ID NOS: 5-21. Most preferably, the mutant recombinant polypeptide comprising a PPR RNA-binding domain will be at least 99% identical to a polypeptide encoded by the sequence of any one of SEQ ID NOS: 5-21.

The invention further provides for an isolated nucleic acid encoding the recombinant polypeptide or the fusion protein of the invention.

Further features of the invention provide for the isolated nucleic acid to have a sequence of any one of SEQ ID NOS: 5-21.

The invention encompasses an isolated nucleic acid encoding the recombinant polypeptide or the fusion protein of the invention that is at least 40% identical; at least 45%; at least 50%; at least 55%; at least 60%; at least 65%; at least 70%; at least 75%; at least 80%; at least 85%; at least 90%; at least 95%; or at least 97% identical; to the sequence of any one of SEQ ID NOS: 5-21. Most preferably, the isolated nucleic acid encoding the recombinant polypeptide or the fusion protein will be at least 99% identical to the sequence of any one of SEQ ID NOS: 5-21.

The invention yet further provides a recombinant vector comprising nucleic acid encoding the recombinant polypeptide or the fusion protein of the invention.

Further features of the invention provide for the nucleic acid of the recombinant vector to have a sequence of the sequence of any one of SEQ ID NOS: 5-21. The invention encompasses a recombinant vector comprising nucleic acid encoding the recombinant polypeptide or the fusion protein of the invention that is at least 40% identical to the sequence of any one of SEQ ID NOS: 5-21. Preferably, the nucleic acid of the recombinant vector will be at least 45%; at least 50%; at least 55%; at least 60%; at least 65%; at least 70%; at least 75%; at least 80%; at least 85%; at least 90%; at least 95%; or at least 97% identical; to the sequence of any one of SEQ ID NOS: 5-21. Most preferably, the nucleic acid of the recombinant vector will be at least 99% identical to the sequence of any one of SEQ ID NOS: 5-21.

The invention extends to a host cell comprising nucleic acid encoding the recombinant polypeptide or the fusion protein of the invention; and for the nucleic acid of the host cell to have a sequence of the sequence of any one of SEQ ID NOS: 5-21.

The invention encompasses a host cell comprising nucleic acid encoding the recombinant polypeptide or the fusion protein of the invention, that is at least 40%; at least 45%; at least 50%; at least 55%; at least 60%; at least 65%; at least 70%; at least 75%; at least 80%; at least 85%; at least 90%; at least 95%; or at least 97% identical to either SEQ ID NO: 1 or SEQ ID NO: 2. Most preferably, the nucleic acid of the host cell will be at least 99% identical to either SEQ ID NO: 1 or SEQ ID NO: 2.

The recombinant polypeptide of the invention or the fusion protein of the invention may further comprise an operable signal sequence such as those known in the art, including but not limited to a nuclear localization signal (NLS), a mitochondrial targeting sequence (MTS) and a secretion signal. The isolated nucleic acid of the invention, the nucleic acid of the recombinant vector of the invention, and the nucleic acid of the host cell of the invention may encode an operable signal sequence such as those known in the art, including but not limited to a nuclear localization signal (NLS), a mitochondrial targeting sequence (MTS), a chloroplast targeting sequence (CTS), a plastid targeting signal, and a secretion signal. The recombinant polypeptide of the invention or the fusion protein of the invention may further comprise a protein tag such as those known in the art, including but not limited to an intein tag, a maltose binding protein domain tag, a histidine tag, a FLAG-tag, a biotin tag, a strepavidin tag, a starch binding protein domain tag, a hemagglutinin tag, and a fluorescent protein tag.

The invention also provides for a composition comprising the recombinant polypeptide of the invention or the fusion protein of the invention or the isolated nucleic acid of the invention or the recombinant vector of the invention.

The invention extends to the use of an effective amount of the recombinant polypeptide of the invention or the fusion protein of the invention or the isolated nucleic acid of the invention or the recombinant vector of the invention in the manufacture of a medicament for regulating gene expression.

The invention further provides for a method of regulating expression of a gene in a cell, the method comprising the step of introducing into the cell a recombinant polypeptide comprising a PPR RNA-binding domain comprising a plurality of consecutively ordered pairs of PPR RNA base-binding motifs operable to bind a target RNA molecule with a target RNA sequence, each pair of PPR RNA base-binding motifs capable of specifically binding to a cytosine, adenine, guanine, or uracil RNA base, wherein the consecutive order of the pairs of PPR RNA base-binding motifs corresponds with the target RNA sequence; and wherein the binding of the recombinant polypeptide to the target RNA alters the expression of the gene.

The method of regulating expression of a gene of a cell may be a method of activating translation, of blocking ribosome binding or ribosome scanning, of regulating RNA splicing, of stimulating RNA cleavage, or of stabilizing the transcript thereby preventing or delaying degradation.

The polypeptides and proteins of the present invention also encompass modified peptides, i.e. peptides, which may contain amino acids modified by addition of any chemical residue, such as phosphorylated or myristylated amino acids.

The invention further provides for a pharmaceutical composition comprising the recombinant polypeptide of the invention or the fusion protein of the invention or the isolated nucleic acid of the invention or the recombinant vector of the invention.

The term “pharmaceutical composition” as used herein comprises the substances of the present invention and optionally one or more pharmaceutically acceptable carriers. The substances of the present invention may be formulated as pharmaceutically acceptable salts. Acceptable salts comprise acetate, methylester, HCl, sulfate, chloride and the like. The pharmaceutical compositions can be conveniently administered by any of the routes conventionally used for drug administration, for instance, orally, topically, parenterally or by inhalation. The substances may be administered in conventional dosage forms prepared by combining the drugs with standard pharmaceutical carriers according to conventional procedures. These procedures may involve mixing, granulating and compressing or dissolving the ingredients as appropriate to the desired preparation. It will be appreciated that the form and character of the pharmaceutically acceptable character or diluent is dictated by the amount of active ingredient with which it is to be combined, the route of administration and other well-known variables. The carrier(s) must be “acceptable” in the sense of being compatible with the other ingredients of the formulation and not deleterious to the recipient thereof. The pharmaceutical carrier employed may be, for example, either a solid or liquid. Exemplary of solid carriers are lactose, terra alba, sucrose, talc, gelatine, agar, pectin, acacia, magnesium stearate, stearic acid and the like. Exemplary of liquid carriers are phosphate buffered saline solution, syrup, oil such as peanut oil and olive oil, water, emulsions, various types of wetting agents, sterile solutions and the like. Similarly, the carrier or diluent may include time delay material well known to the art, such as glyceryl mono-stearate or glyceryl distearate alone or with a wax. The substance according to the present invention can be administered in various manners to achieve the desired effect. Said substance can be administered either alone or in the formulated as pharmaceutical preparations to the subject being treated either orally, topically, parenterally or by inhalation. Moreover, the substance can be administered in combination with other substances either in a common pharmaceutical composition or as separated pharmaceutical compositions. The diluent is selected so as not to affect the biological activity of the combination. Examples of such diluents are distilled water, physiological saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like. A therapeutically effective dose refers to that amount of the substance according to the invention which ameliorate the symptoms or condition. Therapeutic efficacy and toxicity of such compounds can be determined by standard pharmaceutical procedures in cell cultures or experimental animals, e.g., ED50 (the dose therapeutically effective in 50% of the population) and LD50 (the dose lethal to 50% of the population). The dose ratio between therapeutic and toxic effects is the therapeutic index, and it can be expressed as the ratio, LD50/ED50. The dosage regimen will be determined by the attending physician and other clinical factors; preferably in accordance with any one of the methods described above. As is well known in the medical arts, dosages for any one patient depends upon many factors, including the patient's size, body surface area, age, the particular compound to be administered, sex, time and route of administration, general health, and other drugs being administered concurrently. Progress can be monitored by periodic assessment. Specific formulations of the substance according to the invention are prepared in a manner well known in the pharmaceutical art and usually comprise at least one active substance referred to herein above in admixture or otherwise associated with a pharmaceutically acceptable carrier or diluent thereof. For making those formulations the active substance(s) will usually be mixed with a carrier or diluted by a diluent, or enclosed or encapsulated in a capsule, sachet, cachet, paper or other suitable containers or vehicles. A carrier may be solid, semisolid, gel-based or liquid material, which serves as a vehicle, excipient or medium for the active ingredients. Said suitable carriers comprise those mentioned above and others well known in the art, see, e.g., Remington's Pharmaceutical Sciences, Mack Publishing Company, Easton, Pa. The formulations can be adapted to the mode of administration comprising the forms of tablets, capsules, suppositories, solutions, suspensions or the like. The dosing recommendations will be indicated in product labeling by allowing the prescriber to anticipate dose adjustments depending on the considered patient group, with information that avoids prescribing the wrong drug to the wrong patients at the wrong dose.

The invention also provides a system for regulating gene expression comprising
a. a modular set of isolated nucleic acids encoding a plurality of pairs of PPR RNA base-binding motifs, the set including: at least two isolated nucleic acids each encoding a pair of PPR RNA base-binding motif capable of binding to an RNA base;
b. means for annealing the isolated nucleic acids of the modular set in a desired sequence to produce an isolated nucleic acid encoding an expressable recombinant polypeptide comprising a PPR RNA-binding domain having a plurality of consecutively ordered pairs of PPR RNA base-binding motifs; and
c. a target RNA molecule with a target RNA sequence, wherein the consecutive order of the pairs of PPR RNA base-binding motifs corresponds with the target RNA sequence.

Further features of the invention provide for each pair of PPR RNA base-binding motifs to comprise between 30 and 40 amino acids.

The target RNA molecule may be RNA encoding a reporter protein including, but not limited to, his3, β-galatosidase, GFP, RFP, YFP, luciferase, β-glucuronidase, and alkaline phosphatase.

The target RNA molecule may be RNA transcribed from chloroplast and/or mitochondrial genes. The chloroplast and/or mitochondrial genes may be endogenous or exogenous. Furthermore, the target RNA molecule may be derived or expressed by a plant cell, such as, but not limited to, a tobacco plant cell.

Further features of the invention provide for the plurality of pairs of PPR RNA base-binding motifs to comprise between 2 and 40 PPR RNA base-binding motifs, preferably between 8 and 20 PPR RNA base-binding motifs.

Yet further features provide for the PPR RNA-binding domain to comprise a plurality of pairs of PPR RNA base-binding motifs operably linked via amino acid spacers; for such amino acid spacers to include such as those typically used by persons skilled in the art such as, but not limited to, synthetic amino acid spacers, and further for the amino acid spacers to be derived, wholly or in part, from PPR proteins derived from one or more of the group comprising Zea Mays (maize), Oryza sativa (Asian rice), Oryza glaberrima (African rice), Hordeum spp. (Barley), and Arabidopsis spp. (Rockcress) such as Arabidopsis thaliana or any other species harboring PPR proteins. These PPR proteins are given as examples and it will be that these examples are intended for the purpose of exemplification.

The invention extends to a kit for regulating gene expression comprising
a. a modular set of isolated nucleic acids encoding a plurality of pairs of PPR RNA base-binding motifs, the set including: at least two isolated nucleic acids each encoding a pair of PPR RNA base-binding motif capable of specifically binding to an RNA base;
b. means for annealing the isolated nucleic acids of the modular set in a desired sequence to produce an isolated nucleic acid encoding a recombinant polypeptide comprising a PPR RNA-binding domain having a plurality of consecutively ordered pairs of PPR RNA base-binding motifs; and
c. optionally, a target RNA molecule with a target RNA sequence, wherein the consecutive order of the pairs of PPR RNA base-binding motifs corresponds with the target RNA sequence.

Further features of the invention provide for each pair of PPR RNA base-binding motifs to comprise between 30 and 40 amino acids.

The target RNA molecule may be RNA encoding a reporter protein including, but not limited to, his3, β-galatosidase, GFP, RFP, YFP, luciferase, β-glucuronidase, and alkaline phosphatase.

The target RNA molecule may be RNA transcribed from chloroplast and/or mitochondrial genes. The chloroplast and/or mitochondrial genes may be endogenous or exogenous. Furthermore, the target RNA molecule may be derived or expressed by a plant cell, such as, but not limited to, a tobacco plant cell.

Further features of the invention provide for the plurality of pairs of PPR RNA base-binding motifs to comprise between 2 and 40 PPR RNA base-binding motifs, preferably between 8 and 20 PPR RNA base-binding motifs.

Yet further features provide for the PPR RNA-binding domain to comprise a plurality of RNA base-binding motifs operably linked via amino acid spacers; for such amino acid spacers to include those typically used by persons skilled in the art; and further for the amino acid spacers to be derived, wholly or in part, from PPR proteins derived from one or more of the group comprising Zea Mays (maize), Oryza sativa (Asian rice), Oryza glaberrima (African rice), Hordeum spp. (Barley), and Arabidopsis spp. (Rockcress) such as Arabidopsis thaliana. These PPR proteins are given as examples and it will be that these examples are intended for the purpose of exemplification.

The invention also provides a method of identifying a binding target RNA sequence of a PPR RNA-binding domain comprising at least a pair of PPR RNA base-binding motifs operably capable of binding to a target RNA base, the method comprising the steps of:
a. identifying the amino acid at position six of the first PPR motif;
b. identifying the amino acid at position one of the second PPR motif; and
c. assigning to the pair of PPR motifs a binding target RNA base selected from the group comprising adenine (A), guanine (G), cytosine (C), and uracil (U);
wherein the amino acid position six of the first PPR motif is selected from the group consisting of threonine (T), serine (S), and glycine (G), amino acid position one of the second adjacent PPR binding motif is selected from the group comprising asparagine (N), threonine (T), and serine (S), and an adenine (A) RNA base is assigned to the pair of PPR motifs;
wherein the amino acid position six of the first PPR motif is selected from the group consisting of threonine (T), serine (S), glycine (G), and alanine (A), amino acid position one of the second adjacent PPR binding motif is selected from the group comprising aspartic acid (D), threonine (T), and serine (S), and a guanine (G) RNA base is assigned to the pair of PPR motifs;
wherein the amino acid position six of the first PPR motif is threonine (T) or asparagine (N), amino acid position one of the second adjacent PPR binding motif is selected from the group comprising asparagine (N), serine (S), aspartic acid (D), and threonine (T), and a cytosine (C) RNA base is assigned to the pair of PPR motifs; and
wherein the amino acid position six of the first PPR motif is threonine (T) or asparagine (N), amino acid position one of the second adjacent PPR binding motif is selected from the group comprising aspartic acid (D), serine (S), asparagine (N), and threonine (T), and a uracil (U) RNA base is assigned to the pair of PPR motifs.

The method of identifying a target RNA sequence of a PPR RNA-binding domain may comprise the further step of:
d. assigning to each of a plurality of pairs of PPR motifs a binding target RNA base selected from the group comprising adenine (A), guanine (G), cytosine (C), and uracil (U);
wherein the consecutive order of the binding target RNA bases assigned corresponds with the consecutive order of the plurality of pairs of PPR RNA base-binding motifs in the PPR domain, thereby providing the target RNA sequence.

The binding target RNA sequence may be RNA transcribed from chloroplast and/or mitochondrial genes. The chloroplast and/or mitochondrial genes may be endogenous or exogenous. Furthermore, the binding target RNA sequence may be derived or expressed by a plant or plant cell, such as, but not limited to, a tobacco plant or plant cell.

In other words, the method of the invention may be carried out on a plant or plant cell, such as: but not limited to, a tobacco plant or plant cell.

In a preferred embodiment of the invention, the method of identifying a binding target RNA sequence comprises a method of identifying a plant binding target RNA sequence of a plant PPR RNA-binding domain comprising at least a pair of PPR RNA base-binding motifs operably capable of binding to a target RNA base, the method comprising the steps of:
a. identifying the amino acid at position six of the first PPR motif;
b. identifying the amino acid at position one of the second PPR motif; and
c. assigning to the pair of PPR motifs a binding target RNA base selected from the group comprising adenine (A), guanine (G), cytosine (C), and uracil (U);
wherein the amino acid position six of the first PPR motif is selected from the group consisting of threonine (T), serine (S), and glycine (G), amino acid position one of the second adjacent PPR binding motif is selected from the group comprising asparagine (N), threonine (T), and serine (S), and an adenine (A) RNA base is assigned to the pair of PPR motifs;
wherein the amino acid position six of the first PPR motif is selected from the group consisting of threonine (T), serine (S), glycine (G) and alanine (A), amino acid position one of the second adjacent PPR binding motif is selected from the group comprising aspartic acid (D), threonine (T), and serine (S), and a guanine (G) RNA base is assigned to the pair of PPR motifs;
wherein the amino acid position six of the first PPR motif is threonine (T) or asparagine (N), amino acid position one of the second adjacent PPR binding motif is selected from the group comprising asparagine (N), serine (S), aspartic acid (D), and threonine (T), and a cytosine (C) RNA base is assigned to the pair of PPR motifs; and
wherein the amino acid position six of the first PPR motif is threonine (T) or asparagine (N), amino acid position one of the second adjacent PPR binding motif is selected from the group comprising aspartic acid (D), serine (S), asparagine (N), and threonine (T), and a uracil (U) RNA base is assigned to the pair of PPR motifs.

The method of identifying a binding target RNA sequence may further comprise the step of
d. synthesizing a nucleic acid having a sequence comprising the sequence of a plurality of binding target RNA bases assigned in consecutive order to a plurality of PPR motifs.

The synthesized nucleic acid may be introduced into a host cell having the PPR RNA-binding domain using methods typically used by persons skilled in the art. It will be appreciated that such an introduced synthesized nucleic acid sequence either comprises or encodes a target RNA sequence to which the PPR RNA-binding domain is capable of binding. It will also be appreciated that the PPR RNA-binding domain will be capable of binding to the target RNA sequence of the synthesized nucleic acid in similar fashion to the binding of the PPR RNA-binding domain to an endogenous target RNA sequence identified using the method of the invention. Alternatively, the PPR RNA-binding domain may be capable of binding to the target RNA sequence of the synthesized nucleic acid in preference to the endogenous target RNA sequence.

### BRIEF DESCRIPTION OF THE DRAWINGS

Further features of the present invention are more fully described in the following description of several non-limiting embodiments thereof. This description is included solely for the purposes of exemplifying the present invention. It should not be understood as a restriction on the broad summary, disclosure or description of the invention as set out above. The description will be made with reference to the accompanying drawings in which:

FIG. 1 shows alignments between PPR Proteins and Cognate Binding Sites, according to example 1. (A) Statistically optimal alignments between amino acids at positions 6 (blue) and 1′ (red) in PPR10′s PPR motifs and its RNA ligands (italics). PPR10's in vivo footprints are shown at top; the box marks the minimal binding site defined in vitro. Dark green shading indicates experimentally validated matches (FIG. 8). Light green shading indicates significant correlation between position 6 and the purine/pyrimidine class of the matched nucleotide (FIG. 6). Magenta shading indicates significant anti-correlation between position 6 and the purine/pyrimidine class of the matched nucleotide (FIG. 6). Compensatory changes in orthologous protein/RNA pairs are indicated with a star. The PPR motifs are ordered from N to C terminus in the protein, and nucleotides are ordered from 5′ to 3′ in the RNA. The same schemes apply to panels (C) and (D). (B) Structural model illustrating physical plausibility of the cooperation between amino acids at positions 6 and 1′ in nucleotide specification. The model of the PPR10-atpH RNA complex was produced using distance geometry methods as previously described (Fujii S, Bond CS, Small ID (2011) Selection patterns on restorer-like genes reveal a conflict between nuclear and mitochondrial genomes throughout angiosperm evolution. Proc Natl Acad Sci USA 108: 1723-1728). RNA bases were constrained to be within 3 Å of residues 6 and 1′ of helices A and A′ of adjacent motifs. Each PPR motif consists of one “A” and one “B” helix, as marked. (C) Alignments between amino acids at positions 6 and 1′ in PPR motifs of HCF152 and CRP1 and their RNA ligands. The psbH-petB sequence is HCF152's in vivo footprint (Ruwe H, Schmitz-Linneweber C (2012) Short non-coding RNA fragments accumulating in chloroplasts: footprints of RNA binding proteins? Nucleic Acids Res. 40: 3106-3116), within which HCF152 binds in vitro (Zhelyazkova P, Hammani K, Rojas M, Voelker R, Vargas-Suarez M, et al. (2012) Protein-mediated protection as the predominant mechanism for defining processed mRNA termini in land plant chloroplasts. Nucleic Acids Res 40:3092-3105). The petB-petD sequence is a CRP1-dependent in vivo footprint (Zhelyazkova P, Hammani K, Rojas M, Voelker R, Vargas-Suarez M, et al. (2012) Protein-mediated protection as the predominant mechanism for defining processed mRNA termini in land plant chloroplasts. Nucleic Acids Res 40:3092-3105.). The psaC sequence maps within the 70-nt region that most strongly coimmunoprecipitates with CRP1 (Schmitz-Linneweber C, Williams-Carrier R, Barkan A (2005) RNA immunoprecipitation and microarray analysis show a chloroplast pentatricopeptide repeat protein to be associated with the 5′-region of mRNAs whose translation it activates. Plant Cell 17: 2791-2804). (D) Alignments between amino acids at positions 6 and 1′ in PPR motifs of the RNA editing factors OTP82, CRR22 and CRR4 and their RNA targets (Okuda K, Shikanai T (2012) A pentatricopeptide repeat protein acts as a site-specificity factor at multiple RNA editing sites with unrelated cis-acting elements in plastids. Nucleic Acids Res. 40: 5052-506; Okuda K, Nakamura T, Sugita M, Shimizu T, Shikanai T (2006) A pentatricopeptide repeat protein is a site recognition factor in chloroplast RNA editing. J Biol Chem 281: 37661-37667). Minimal binding sites determined in vitro are boxed. The edited C (magenta) is the last nucleotide in each case. The type of PPR motif, either P, L or S, is indicated above. Only matches involving P or S motifs are shaded, as L motifs cannot be accommodated within the code developed here;

FIG. 2 shows alignments of PPR10 to the PPR10 RNA footprint ranked by p-value, according to example 2. The table shows the top 100 alignments out of the 29400 possible. The two alignments shaded in yellow correspond to the alignments depicted in FIG. 1. Orientation: forward indicates N→C, 5′-3′; reverse indicates N→C, 3′-5′. Offset: distance from start of RNA sequence to first PPR motif. Gap position: nucleotide at which gap introduced between protein motifs. Gap length: length of gap in nucleotides. 17-mer: position (from 1 to 35) within the PPR motifs used to constitute the 17-mer sequence of amino acids used for the alignment. P-value: probability that amino acids and nucleotides are arranged independently of each other, as calculated by Fisher's Exact Test. None of the 29400 alignments exceed the threshold for significance at the 5% level if a threshold corrected for the total number of tests is used (5% threshold using the \check{S}idák correction=1.74E-06);

FIG. 3 shows a table of Correlations between amino acids at specific positions within PPR motifs and aligned nucleotides, according to example 2. Contingency tables (amino acids versus nucleotides) were constructed from the alignments in FIG. 1 and FIG. 9. Each 20×4 table was tested for independent assortment of amino acids and nucleotides using a chi-squared test (after first removing any empty rows from the table). P-values from the tests are shown in the table, with those values that are significant for both P and S motifs highlighted (a 1% significance threshold was used, corrected for multiple tests using the \check{S}idák correction). Rows: amino acid positions within the motifs. Columns: 0 indicates the motif aligned with the nucleotide, −1 the preceding motif, +1 the following motif;

FIG. 4 shows amino acid representation at each position of PPR motifs that align with A, G, C, or U bases, according to example 2. Motif pairs from PPR10, HCF152, CRP1 and 37 RNA editing factors flanking the indicated nucleotide were used to construct sequence logos. Each logo shows the first fifteen positions of the P-type motif containing position 6, a gap, and then the first 5 positions of the following motif. 74, 48, 96 and 126 motif pairs were used to generate the A, G, C and U logos, respectively. The editing factor alignments used to generate the logos are shown in FIG. 9; the other alignments are shown in FIG. 1;

FIG. 5 shows nucleotides that align with the most frequent combinations of amino acids at positions 6 and 1′, according to example 2. Nucleotides aligned with each 6/1′ combination in the alignments in FIG. 9 were used to construct sequence logos. Only P motifs were used in this analysis. Each logo shows the aligned nucleotide (0) and the preceding (−1) and succeeding (+1) nucleotides. 25, 23, 102, 86 and 16 alignments were used to generate the T₆N_{1′}, T₆D_{1′}, N₆D_{1′}, N₆N_{1′} and N₆S_{1′} logos, respectively;

FIG. 6 shows correlations between amino acids at positions 6, 1′ and aligned nucleotides, according to example 2. The tables show frequencies of co-occurrence of amino acids and nucleotides from the alignments in FIGS. 1 and 9. (A) P motifs, positions 6, 1′ versus each nucleotide. (B) S motifs, positions 6, 1′ versus each nucleotide. (C) P motifs, position 6 versus purines (R), pyrimidines (Y). (D) S motifs, position 6 versus purines (R), pyrimidines (Y). P-values were calculated using G-tests. P-values in A and B are for the most positively correlated nucleotide. Significance was evaluated at 5% allowing for multiple testing (using the \check{S}idák correction). Green shading indicates significantly correlated, magenta shading indicates significantly anti-correlated;

FIG. 7 shows the frequency of 6,1′ combinations in Arabidopsis PPR proteins, according to example 2. The most frequent combinations are shown (all those observed more than 30 times). Only tandem pairs of motifs (5362 in total) were considered in this analysis, where the first motif was either a P or S motif. Combinations observed in P motifs are shown in blue, those in S motifs in green;

FIG. 8 shows gel mobility shift assays validating amino acid codes for specifying PPR Binding to A, G, C, or U (A) Summary of rPPR10 variants, according to example 2. The same amino acids at positions 6 and 1′ were introduced into the sixth and seventh PPR motifs in PPR10, whose wild-type sequences are shown above. The RNAs used for binding assays are shown below. (B) Gel mobility shift assays with the wild-type RNA, or variants with nucleotides four and five substituted with either GG, AA, UU, or CC. (C) Binding curves of the NN, ND, and NS PPR10 variants with the UU and CC substituted RNAs;

FIG. 9 shows alignments of PPR editing factors to their target sites, according to example 2. For each factor, the name of the protein and its editing site are listed, then successively the types of PPR motif, the amino acids at position 6, the amino acids at position 1′, an indication of the degree to which these amino acids ‘match’ the RNA using the code developed in this work, and lastly the RNA sequence (in lower case). ‘:’ and ‘.’ indicate experimentally validated (see FIG. 8) and computationally predicted (see FIG. 4) matches, respectively. Mismatches are indicated by ‘x’. All proteins are aligned such that the C-terminal S motif aligns with the nucleotide at -4 with respect to the edited C (indicated in upper case);

FIG. 10 shows that PPR10 bound in a 5′ UTR blocks translation by 80S (eukaryotic) ribosomes in vitro, according to example 2. An mRNA encoding luciferase with a 5′UTR either containing two PPR10 binding sites, or containing the same nucleotide content in a shuffled order was incubated in a wheat germ translation extract for either 30 or 60 minutes. Recombinant PPR10 was added to a subset of the reactions. The presence of PPR10 and luciferase was detected by western blotting. The translation of the mRNA harboring the PPR10 binding sites in the 5′UTR was specifically repressed by recombinant PPR10;

FIG. 11 shows gel mobility shift assays with the SN variant, according to example 2; The experimental design was that the same as that for the experiment in FIG. 8;

FIG. 12 shows gel mobility shift assays with the TT variant, according to example 2; The experimental design was that the same as that for the experiment in FIG. 8;

FIG. 13 shows gel mobility shift assays with the AD variant, according to example 2; The experimental design was that the same as that for the experiment in FIG. 8;

FIG. 14 shows gel mobility shift assays with the TS variant according to example 2; The experimental design was that the same as that for the experiment in FIG. 8;

FIG. 15 shows alignments of PPR editing factors to their target sites according to example 3. For each factor, the name of the protein and its editing site are listed, then successively the types of PPR motif, the amino acids at position 6, the amino acids at position 1′, an indication of the degree to which these amino acids ‘match’ the RNA using the code developed in this work, and lastly the RNA sequence (in lower case). ‘:’ and indicate experimentally validated (see FIG. 8) and computationally predicted (see FIG. 4) matches, respectively. Mismatches are indicated by ‘x’. All proteins are aligned such that the C-terminal S motif aligns with the nucleotide at −4 with respect to the edited C (indicated in upper case).

SEQ ID NO: 1 is the amino acid sequence of PPR repeats 6, 7, and 8 of PPR10 var (T,D).

SEQ ID NO: 2 is the amino acid sequence of PPR repeats 6, 7, and 8 of PPR10 var (T,N).

SEQ ID NO: 3 is the amino acid sequence of PPR repeats 6, 7, and 8 of PPR10 wild-type.

SEQ ID NO: 4 is the amino acid sequence of wild-type PPR10.

SEQ ID NO: 5 is the DNA sequence of the primer used to prepare a TD variant with a G mutation.

SEQ ID NO: 6 is the DNA sequence of the primer used to prepare the TD variant with a C mutation.

SEQ ID NO: 7 is the DNA sequence of the primer used to prepare another TD variant with a C mutation.

SEQ ID NO: 8 is the DNA sequence of the primer used to prepare another TD variant with a G mutation.

SEQ ID NO: 9 is the DNA sequence of the primer used to prepare another TD variant with a G mutation.

SEQ ID NO: 10 is the DNA sequence of the primer used to prepare a TN variant with a T mutation.

SEQ ID NO: 11 is the DNA sequence of the primer used to prepare a TN variant with an A mutation.

SEQ ID NO: 12 is the DNA sequence of the primer used to prepare another TN variant with an A and C mutation.

SEQ ID NO: 13 is the DNA sequence of the primer used to prepare another TN variant with a G and T mutation.

SEQ ID NO: 14 is the DNA sequence of the primer used to prepare a NN variant with a double A mutation.

SEQ ID NO: 15 is the DNA sequence of the primer used to prepare a NN variant with a double T mutation.

SEQ ID NO: 16 is the DNA sequence of the primer used to prepare a ND variant with a G mutation.

SEQ ID NO: 17 is the DNA sequence of the primer used to prepare a ND variant with a C mutation.

SEQ ID NO: 18 is the DNA sequence of the primer used to prepare a NS variant with an AGC mutation.

SEQ ID NO: 19 is the DNA sequence of the primer used to prepare a NS variant with an GCT mutation.

SEQ ID NO: 20 is the DNA sequence of the primer used to prepare a NS variant with an AGC mutation.

SEQ ID NO: 21 is the DNA sequence of the primer used to prepare a NS variant with an GCT mutation.

Throughout this specification, unless the context requires otherwise, the word “comprise” or variations such as “comprises” or “comprising”, will be understood to imply the inclusion of a stated integer or group of integers but not the exclusion of any other integer or group of integers.

### DESCRIPTION OF EMBODIMENTS

Briefly, the inventors of the present application have identified the critical amino acid residues within pentatricopeptide repeat (PPR) motifs whose modification can alter sequence-specific binding of RNA, and particular combinations of residues that will recognise each RNA base. The inventors have identified particular combinations of amino acid residues within PPR motifs that recognise each of the 4 RNA bases and the determination of the relative polarity of the RNA and PPR tract in the PPR-RNA complex. The invention may be used to design a PPR protein to recognize and bind a desired RNA target sequence.

The inventors used connotation or methods to infer a code for nucleotide recognition involving 2 amino acids in each repeat, validating this code by recoding a PPR protein to bind novel RNA sequences in vitro. Using this approach, the inventors have shown for the first time that PPR tracts recognize RNA via a modular 1-PPR motif/1-nt mechanism, and have deciphered a “code” for RNA recognition. The inventors have also shown that binding must be parallel, and that a successful code works with the assumption of parallel orientation of PPR and RNA. The inventors have further shown that 1:1 correspondence and intercalation are both true for PPR-RNA complexes. The inventors have shown that PPR motifs can be designed to bind either A, G, U>C, or U=C by recoding a PPR protein to bind non-native RNA sequences. These results do not agree with the model put forward in a recent paper by a Japanese group (Kobayashi, K. et al (2011) Nucleic Acids Res, doi: 10.1093/nar/gkr1084). The molecular recognition mechanism by which the inventors show the binding between PPR tracts and RNA differs from previously described RNA-protein recognition modes. It is an advantage of the invention that evolutionary plasticity of the PPR family facilitates redesign of these proteins according to the parameters identified by the inventors for new sequence binding specificities and functions.

### EXAMPLE 1

### Introduction

Models for sequence-specific RNA recognition by PPR tracts were developed, focussing on the maize protein PPR10. PPR10 consists of 19 PPR motifs and little else. PPR10 localizes to chloroplasts, and binds two different RNAs via cis-elements with considerable sequence similarity. PPR10 serves to position processed mRNA termini and stabilize adjacent RNA segments in vivo by blocking exoribonucleases intruding from either direction.

### Materials and Methods

Expression of rPPR10

rPPR10 and its variants were expressed in E. coli and purified as described previously (Pfalz, J., Bayraktar, O., Prikryl, J., and Barkan, A. (2009). EMBO J 28, 2042-2052). In brief, mature PPR10 (i.e. lacking the plastid targeting peptide) was expressed as a fusion to maltose binding protein (MBP), purified by amylose affinity chromatography, separated from MBP by cleavage with TEV protease, and further purified by gel filtration chromatography in 250 mM NaCl, 50 mM Tris-HCl pH 7.5, 5 mM 13-mercaptoethanol. The elution peak was diluted in the same buffer for AUC, or dialyzed against 400 mM NaCl, 50 mM Tris-HCl pH 7.5, 5 mM β-mercaptoethanol, 50% glycerol prior to use in RNA binding assays.

PPR10 variants were obtained by PCR-mutagenesis using the following primers (lower case indicates mutations):

TD Variant:
(SEQ ID NO: 5)
5′ GGTCTGTTGCCAgACGCATTCACG;
(SEQ ID NO: 6)
5′ CGTGAATGCGTcTGGCAACAGACC;
(SEQ ID NO: 7)
5′ GCTGTGACGTACAcCGAGCTCGCCGGAACG;
(SEQ ID NO: 8)
5′ CGTTCCGGCGAGCTCGgTGTACGTCACAGC;
(SEQ ID NO: 9)
5′ CACCTGGAGCAACGCGgTGTACGTGACGACGCAC.
TN Variant:
(SEQ ID NO: 10)
5′ CGTGAATGCGTtTGGCAACAGACCC;
(SEQ ID NO: 11)
5′ GGGTCTGTTGCCAaACGCATTCACG;
(SEQ ID NO: 12)
5′ GAACGGCTGCCAGCCAaAcGCTGTGACGTAC;
(SEQ ID NO: 13)
5′ CGgTGTACGTCACAGCgTtTGGCTGGCAGCCG.
NN Variant:
(SEQ ID NO: 14)
5′ GGAGCAGAACGGCTGCCAGCCAaacGCTGTGACG;
(SEQ ID NO: 15)
5′ CGTCACAGCgttTGGCTGGCAGCCGTTCTGCTCC.
ND Variant:
(SEQ ID NO: 16)
5′ GGTCTGTTGCCAgACGCATTCACG;
(SEQ ID NO: 17)
5′ CGTGAATGCGTcTGGCAACAGACC.
NS Variant:
(SEQ ID NO: 18)
5′ GCTGCCAGCCAagcGCTGTGACG;
(SEQ ID NO: 19)
5′ CGTCACAGCgctTGGCTGGCAGC;
(SEQ ID NO: 20)
5′ GTCTGTTGCCAagcGCATTCACGTACAACACC;
(SEQ ID NO: 21)
5′ GGTGTTGTACGTGAATGCgctTGGCAACAGAC.

### Statistical Analysis of PPR/RNA Alignments

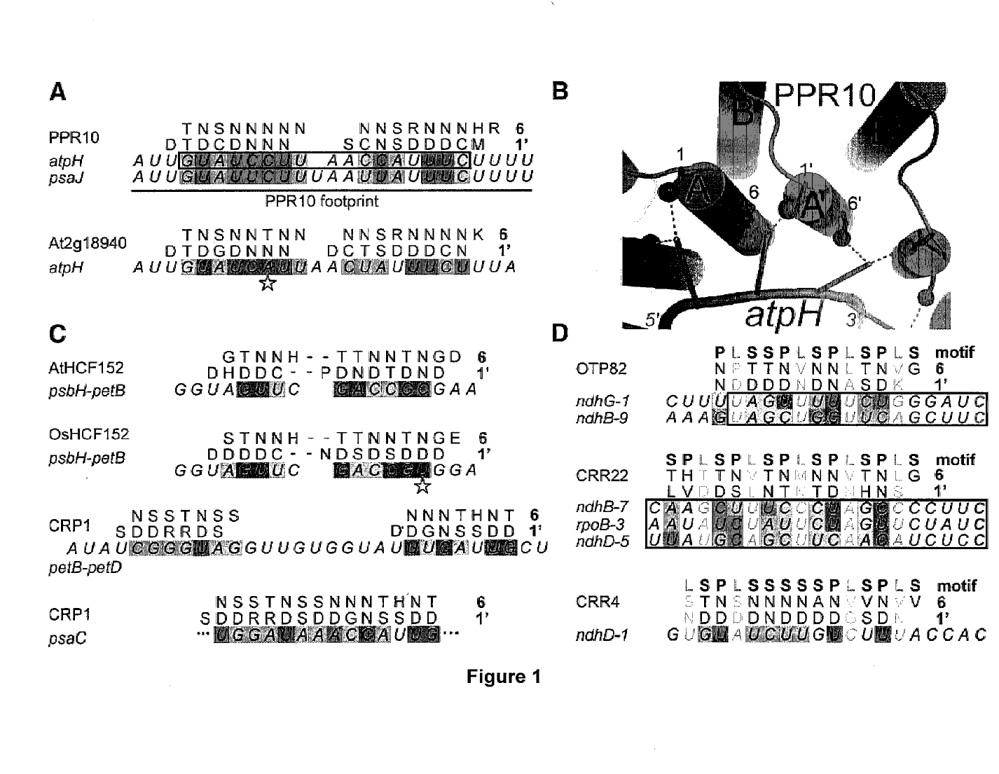

The alignment of PPR10 to its atpH binding site was generated de novo as follows. Thirty-five 17-mers were constructed, each corresponding to the amino acids at a specific position within the 17 sequential PPR motifs in PPR10's interior. Terminal PPR motifs were excluded, as they have distinct properties that may adapt them to their terminal position. These 17 motifs can be arranged in 420 different ways on the 24-nucleotides that are protected by PPR10, assuming that all the motifs contact the RNA sequentially but not necessarily contiguously, and permitting gaps of any length at any position. The number of arrangements is doubled if both polarities of the protein on the RNA are considered. For each of the 840 arrangements, contingency tables were constructed for each of the 35 17-mers, scoring the number of co-occurrences of each possible amino acid/nucleotide pair (i.e. a total of 2940020x4 tables). Fisher's Exact Test was used to test for independence of amino acid and nucleotides classes, as implemented in R version 2.14.2 by fisher test. The tables were ranked by p-value. The top ranked alignment (1/29400) was for position 1. The best alignment for position 6 was also retained (ranked 71/29400). No other highly ranked alignments were physically compatible with the motif arrangement required for the alignment shown in FIG. 1A. (i.e. contained a gap of the same length in the same place). The FIG. 1A alignments are empirically supported by the boundaries of the PPR10 footprint and minimal binding site, by covariations among PPR10 orthologs and their binding sites, by natural variation in the central region of PPR10's two native binding sites, and by binding affinities of PPR10 for variant atpH sites with various insertions and point mutations.

### Gel Mobility Shift Assays

Gel mobility shift assays and K_{d} calculations were performed as described previously (Prikryl, J., Rojas, M., Schuster, G., and Barkan, A. (2011) Proc Natl Acad Sci USA 108, 415-420), using radiolabeled synthetic RNAs at 15 pM and protein at 0, 5, 10, and 20 nM, unless otherwise indicated.

### Results

### Modeling the Polarity and Register of a PPR10-RNA Complex Suggested an Amino Acid Code for RNA Recognition

The minimal PPR10 binding site in the atpH 5′-UTR spans 17-nt and PPR10 leaves a ribonuclease-resistant footprint spanning ˜24 nucleotides (Prikryl, J., Rojas, M., Schuster, G., and Barkan, A. (2011) Proc Natl Acad Sci USA 108, 415-420) (FIG. 1A). To identify specificity determining amino acids, correlations were sought between the amino acid residues at each position of PPR10's PPR motifs and the bases within its footprint. The RNA was modeled in parallel to the protein (i.e. 5′-end aligned with N-terminus) due to the organization of PPR proteins that specify sites of RNA editing: such proteins have an N-terminal PPR tract and a C-terminal domain that is required for editing, and they bind cis-elements that are 5′ of the edited sites. It was further assumed that all motifs would contact an RNA base, but not necessarily contiguously.

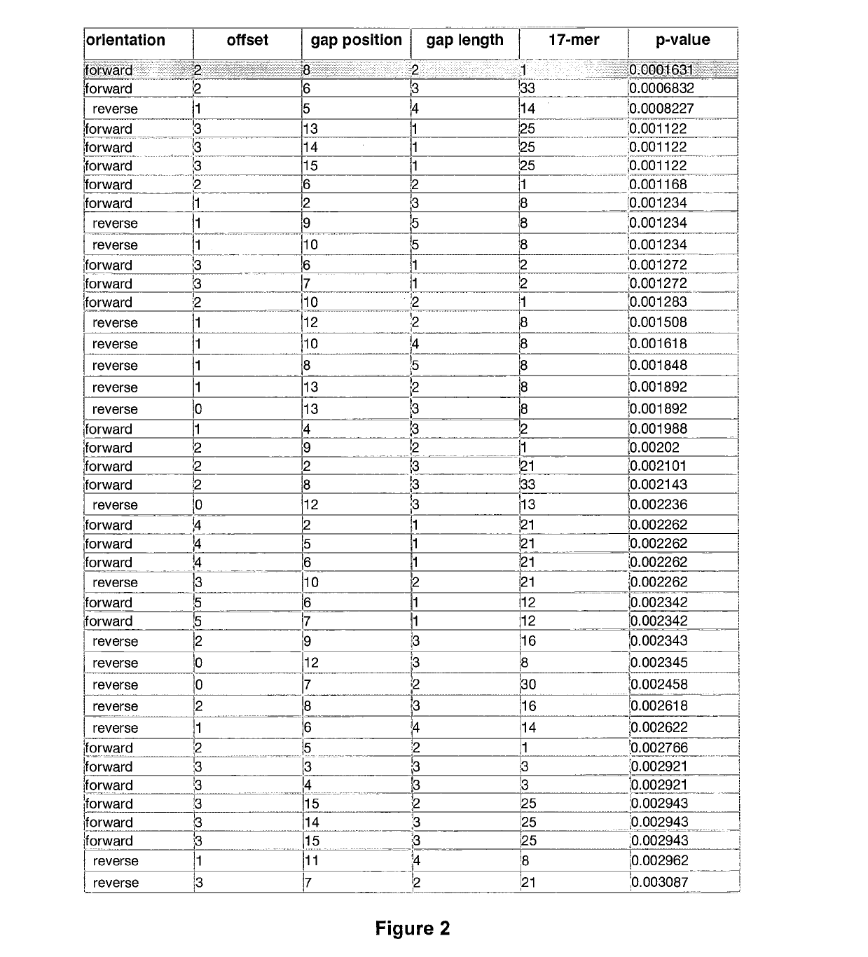

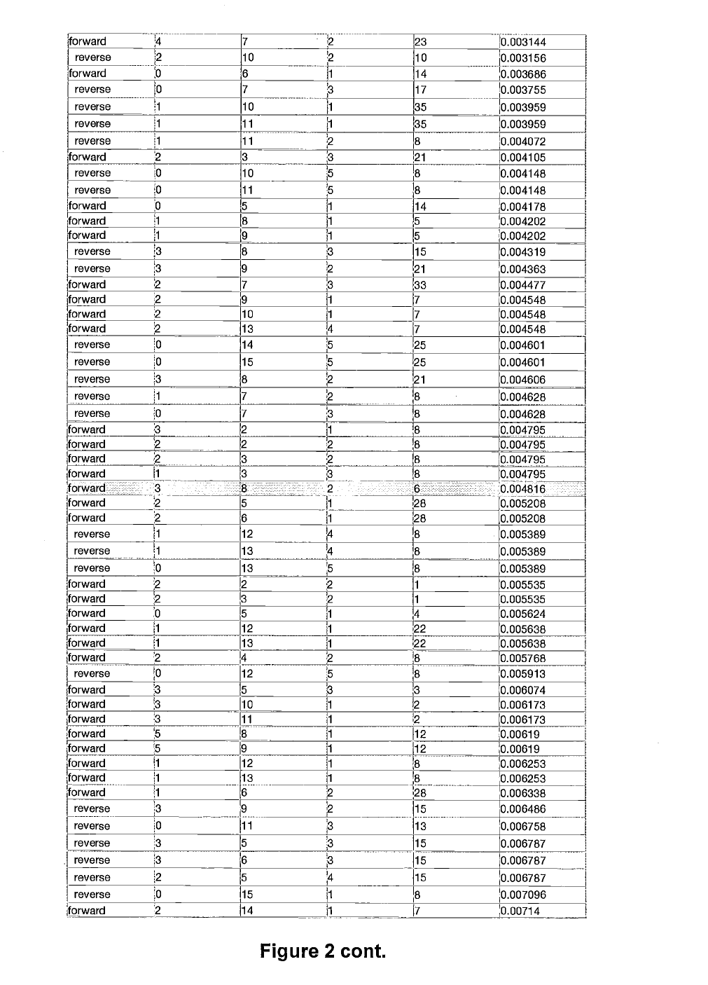

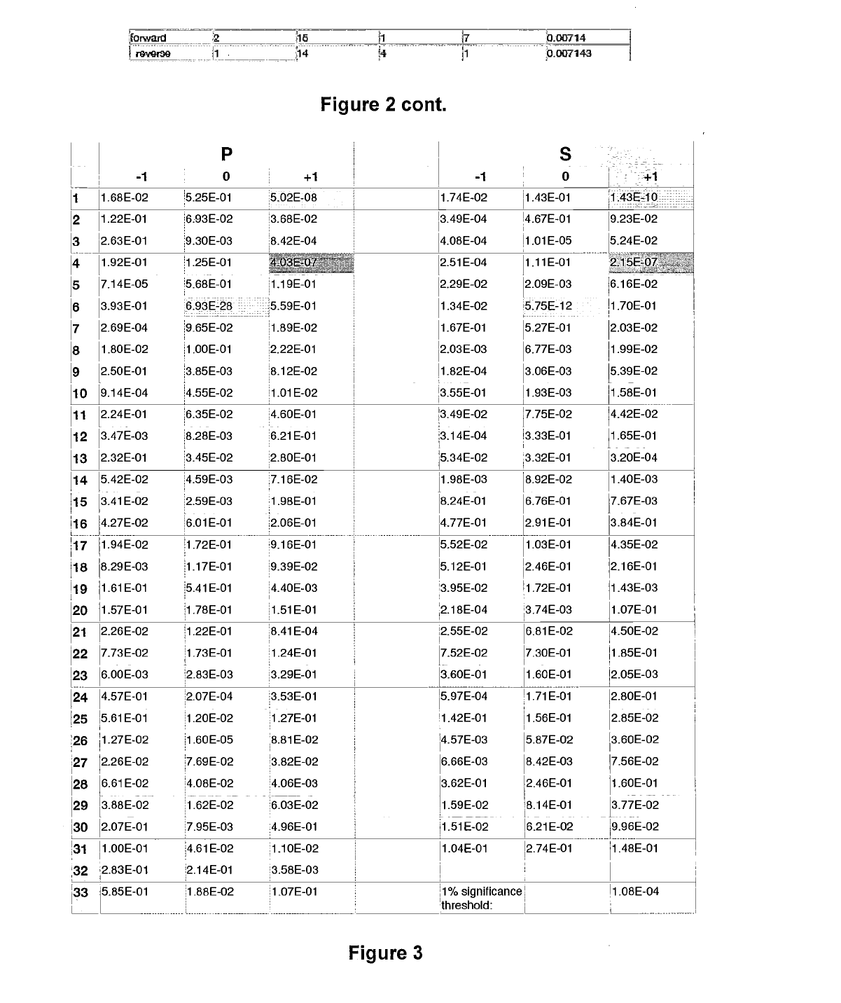

Given these constraints, there are 420 possible arrangements of PPR10's PPR motifs in contact with its RNA footprint (see Materials and Methods section). One of these arrangements showed strong correlations between the RNA base and the amino acids found at positions 1 and 6 (FIG. 1A, FIG. 2).The alignment to amino acid 6 is offset by one nucleotide from the alignment to amino acid 1, such that the base that correlates with position 6 of motif n also correlates with position 1 of the n+1 motif; hereafter this position is referred to as 1′, to distinguish it from position 1 in motif n. This offset is physically plausible (FIG. 1 B), and it is supported by an in vitro analysis of a pair of PPR motifs. The optimal alignment contains a gap that breaks the protein-RNA duplex into two segments. The gap corresponds with the position of a single nucleotide insertion in PPR10′s psaJ binding site (FIG. 1A), providing evidence for relaxed selection in this region of the binding site. This alignment highlights the following correlations: every N₆ aligns with a pyrimidine, each purine corresponds to S₆ or T₆, and every D_{1′} aligns with a U. These correlations are maintained by covariation when the orthologous protein and binding site in Arabidopsis is considered (FIG. 1A).

These correlations were extended by analysis of the PPR protein HCF152 (Meierhoff, K., Felder, S., Nakamura, T., Bechtold, N., and Schuster, G. (2003) Plant Cell 15, 1480-1495), which binds to sequences within its 17-nt footprint in the chloroplast psbH-petB intergenic region (Ruwe, H., and Schmitz-Linneweber, C. (2011). Nucleic Acids Res; Zhelyazkova, P., Hammani, K., Rojas, M., Voelker, R., Vargas-Suarez, M., Borner, T., and Barkan, A. (2011) Nucleic Acids Res Epub December 8). When HCF152's 13 PPR motifs were compared with this sequence, the optimal alignment spanned 12 nucleotides and preserved the correlations observed for PPR10 (FIG. 1C). Furthermore, this alignment is maintained through covariation in rice (FIG. 1C). The maize protein CRP1 further strengthens these correlations. CRP1 leaves a ˜30-nt footprint in the chloroplast petB-petD intergenic region (Barkan, A., Walker, M., Nolasco, M., and Johnson, D. (1994) EMBO J 13, 3170-3181; Zhelyazkova, P., Hammani, K., Rojas, M., Voelker, R., Vargas-Suarez, M., Borner, T., and Barkan, A. (2011) Nucleic Acids Res Epub December 8). CRP1′s 14 PPR motifs can be aligned within this footprint in a manner that retains the correlations noted above (FIG. 1C). Similar to the PPR10 alignments, the CRP1 alignment involves 7 contiguous matches at each end, with “unpaired” nucleotides in the central region. Notably, the PPR10, HCF152, and CRP1 alignments are all placed very similarly within their RNAse-resistant footprints, as is to be expected given that each protein blocks access by the same exonucleases in vivo. Finally, an alignment that follows the same rules can be made between CRP1 and a sequence in the psaC 5′-UTR that maps within the 70-nt segment that is most strongly enriched in CRP1 coimmunoprecipitations (Schmitz-Linneweber, C., Williams-Carrier, R., and Barkan, A. (2005) Plant Cell 17, 2791-2804) (FIG. 1C).

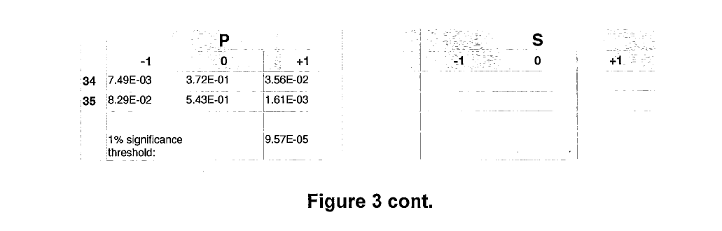

PPR proteins can be separated into two classes, denoted P and PLS. PPR10, HCF152, and CRP1 are examples of P-class proteins, which contain tandem arrays of 35 amino acid PPR motifs. Members of this class have been implicated in RNA stabilization, processing, splicing, and translation. PLS-class proteins contain alternating canonical “P” motifs, and variant ‘long’ and ‘short’ PPR motifs (Lurin, C., Andres, C., Aubourg, S., Bellaoui, M., Bitton, F., Bruyere, C., Caboche, M., Debast, C., Gualberto, J., Hoffmann, B., et al. (2004) Plant Cell 16, 2089-2103), and typically function in RNA editing. PPR editing factors can be aligned to sequences upstream of the edited nucleotide such that the amino acids at position 6 of the ‘P’ motifs and the amino acids at position 1′ of the following motif correlate with the matched nucleotide in a similar manner to that found for the P-class proteins (FIG. 1D). Importantly, the editing factors can all be aligned such that their C-terminal motif is at the same distance from the edited cytidine residue. This not only explains how the target C is defined, it allows the motif-nucleotide correlations in the editing factors to be evaluated without using them to make the alignment. Correlations between the aligned base and the amino acids at positions 6, and 1′ are highly significant across all alignments for both ‘P’ and ‘S’ motifs (FIG. 3). Apart from these two positions, only the amino acid at 4′ is also significantly correlated with the aligned nucleotide.

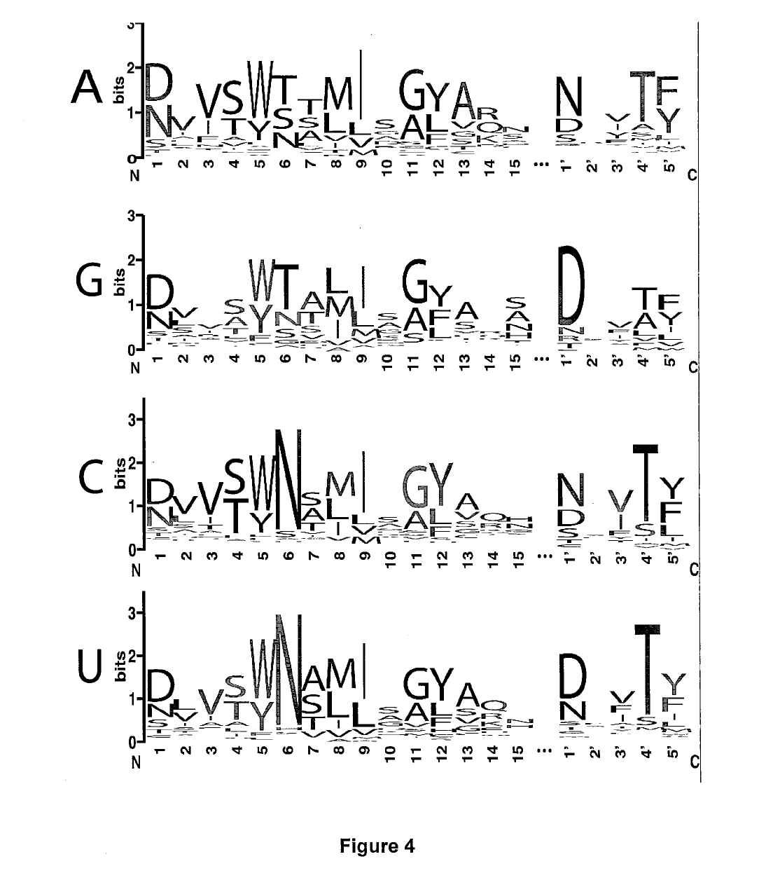

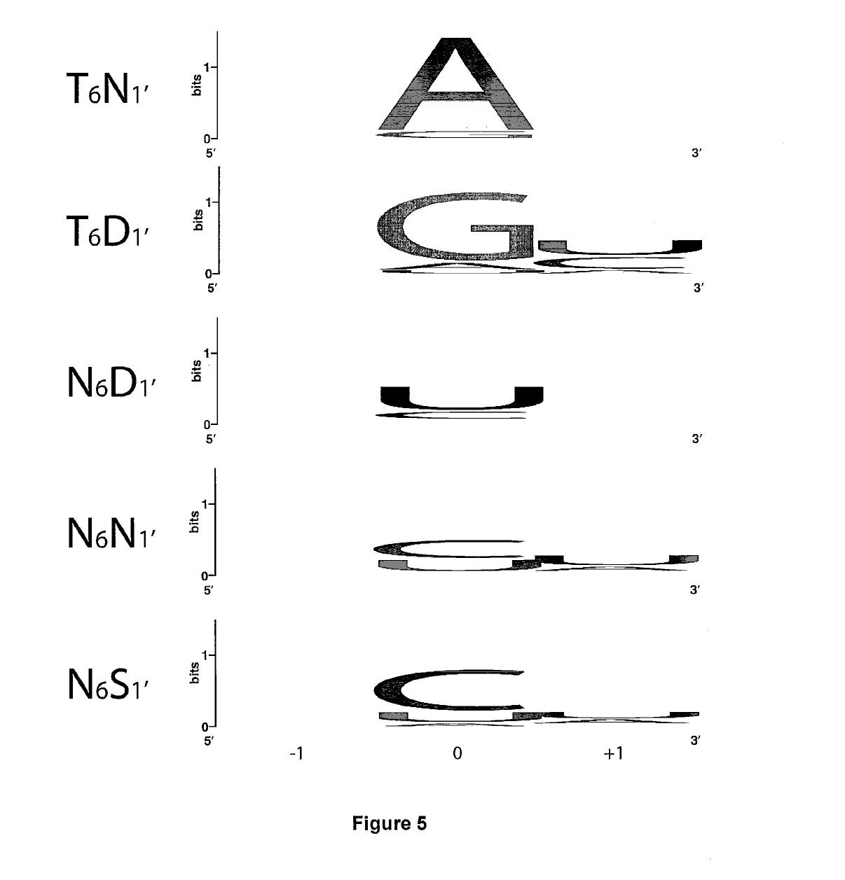

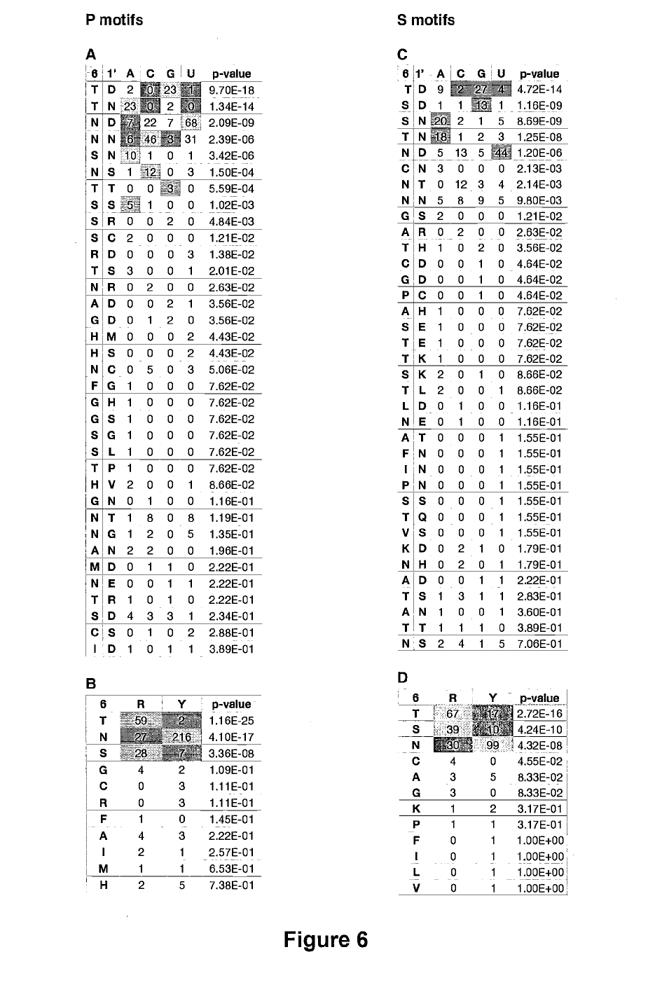

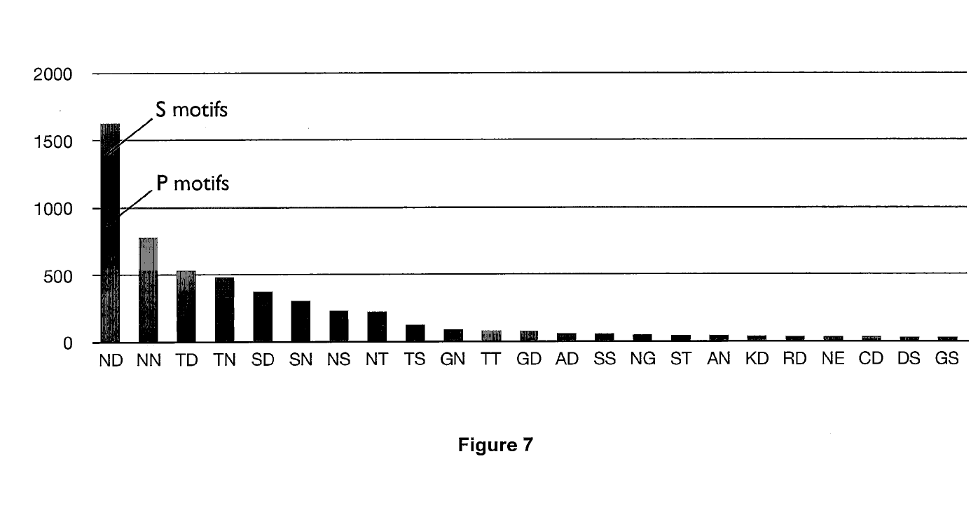

Sequence logos constructed from PPR motif pairs aligned with either A, G, C, or U are shown in FIGS. 4 and 5. From these alignments, a set of rules was derived to represent a combinatorial amino acid code for nucleotide recognition by PPR motifs: T₆D_{1′}=G; T/S₆N_{1′}=A; N₆D_{1′}=U; N₆N/S_{1′}=C. The diversity of amino acid combinations at these positions implies that the code may be degenerate (FIG. 6). However, the above-mentioned amino acid combinations are the most commonly observed, and together represent 64% of all canonical PPR motif pairs in Arabidopsis and rice (FIG. 7).

### Confirmation of a Code by Recoding PPR10 to Bind New RNA Sequences

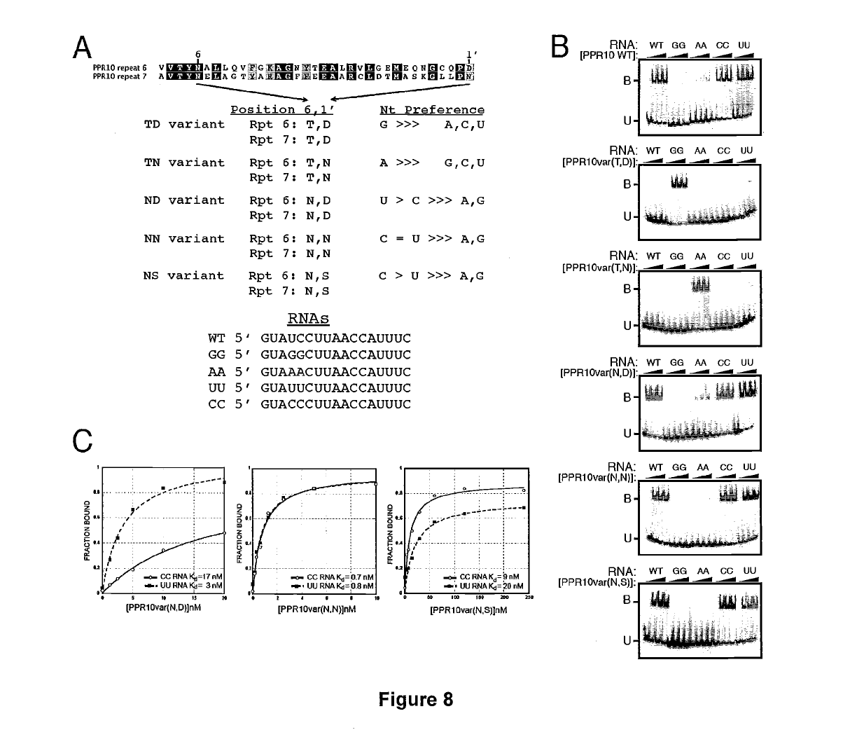

To test whether the correlations between amino acid identities at PPR positions 6 and 1′ and the associated nucleotide reflect a recognition code, a set of PPR10 variants was generated in which residues (6, 1′) in a pair of adjacent repeats (motifs 6 and 7) were modified to either T₆D_{1′}, T₆N_{1′}, N₆D_{1′}, or N₆N_{1′}, or N₆S_{1′} (FIG. 8A). This model aligns PPR10 repeats 6 and 7 with U and C nucleotides, respectively. PPR10 does not bind significantly to RNA in which these nucleotides are substituted with either AA or GG (FIG. 8B). A PPR10 variant in which motifs 6 and 7 were modified to (T,D) did not bind to the wild-type RNA, but bound with high affinity to RNA with the GG substitution. Likewise, the variant in which these motifs were modified to (T,N) did not bind to wild-type RNA, but bound with high affinity to RNA with the AA substitution. Neither variant bound significantly to any of the other substituted RNAs. These results confirmed the proposed polarity and register of the PPR10/RNA complex, and show that (T,D) and (T,N) at positions (6, 1′) are highly specific for binding G and A, respectively.

The (N,D), (N,N) and (N,S) combinations at (6, 1′) correlate with recognition of pyrimidines (FIG. 5 and FIG. 6). As predicted, PPR10 variants with these amino acid combinations strongly favored binding to pyrimidine-substituted RNAs (FIG. 7B). The (N,D) variant bound the U and C substituted RNAs with K_{d}s of ˜3 nM and 17 nM, respectively, indicating a clear preference for U over C (FIG. 8C). Conversely, the (N,S) variant favored C over U, albeit only slightly (K_{d}s of 9 nM and 20 nM for the C and U substituted RNAs, respectively). The (N,N) variant is less discriminating, binding the U and C substituted RNAs with similar affinities (FIG. 8C).

Results presented here provide strong evidence that PPR tracts bind RNA in a parallel orientation via a modular recognition mechanism, with nucleotide specificity relying primarily on the amino acid identities at positions 6 and 1′ in each repeat. Modification of amino acids at these positions in the context of two adjacent PPR motifs was sufficient to change the nucleotide preference, suggesting that other amino acid positions make no more than a small contribution to nucleotide specificity. Position 4′ correlates weakly with the aligned nucleotide, but threonine is preferred at 4′ for all four nucleotides (FIG. 4) and the effect of any other amino acid at this position was not investigated. Although similar in concept to Puf/RNA recognition, PPR/RNA complexes have the opposite polarity to PUF/RNA complexes and involve distinct and different amino acid combinations. The polarity and code demonstrated herein for PPR/RNA interactions differs from those proposed by Kobayashi et al. (Kobayashi K, Kawabata M, Hisano K, Kazama T, Matsuoka K, et al. (2012) Identification and characterization of the RNA binding surface of the pentatricopeptide repeat protein. Nucleic Acids Res 40: 2712-2723), who concluded that the PPR protein HCF152 binds anti-parallel to an A-rich RNA sequence. This model was based on a shallow HCF152 SELEX dataset, from which similarities were sought to a presumed HCF152 binding site that was recently shown not to bind HCF152 with high affinity (Zhelyazkova P, Hammani K, Rojas M, Voelker R, Vargas-Suarez M, et al. (2012) Protein-mediated protection as the predominant mechanism for defining processed mRNA termini in land plant chloroplasts. Nucleic Acids Res 40:3092-3105).

The results set out herein define a combinatorial two-amino acid code for specifying the binding of a PPR motif to either A, G, U>C, C>U, or U=C. This code facilitates engineering of PPR tracts to bind a wide variety of RNA sequences.

The alignments of P-class PPR proteins to their cognate RNAs described herein include contiguous duplexes consisting of no more than nine motifs and 8 nucleotides. The number of contiguous interactions between helical repeats and RNA bases may be constrained by the minimum distance between parallel alpha helices. The minimum theoretical helix-helix distance is c. 9.5 Å. In contrast, adjacent nucleotides in Put RNA complexes are 7 Å apart, close to the maximally extended conformation, and resulting in a distance mismatch that is only partially accommodated by curvature of the RNA-binding surface.

PPR tracts may offer functionalities beyond those achievable with engineered Puf domains due to their more flexible architecture. Unlike Puf domains, whose 8-repeat organization is conserved throughout the eucaryotes, natural PPR proteins have between 2 and ˜30 repeats. The unusually long surface for RNA interaction that is presented by long PPR tracts has the potential to sequester an extended RNA segment.

### EXAMPLE 2

### Materials and Methods

### In Vitro Translation

An mRNA transcript comprising the coding region of luciferase cloned downstream from two PPR10 binding sites was prepared according to standard techniques known in the art. A control mRNA transcript comprising the coding region of luciferase cloned downstream from two spacer sequences which did not comprise a PPR10 binding site was also prepared according to standard techniques. A wheat germ in vitro translation extract was used in an in vitro translation reaction, the products of which were separated by SDS page and transferred to nitrocellulose by Western blotting techniques known in the art. The Western blots were probed using anti-PPR 10 and anti-luciferase antibodies according to techniques known in the art.

### Gel Mobility Shift Assays

Gel mobility shift assays are carried out according to the methods described in Example 1.

### Results

### In Vitro Translation

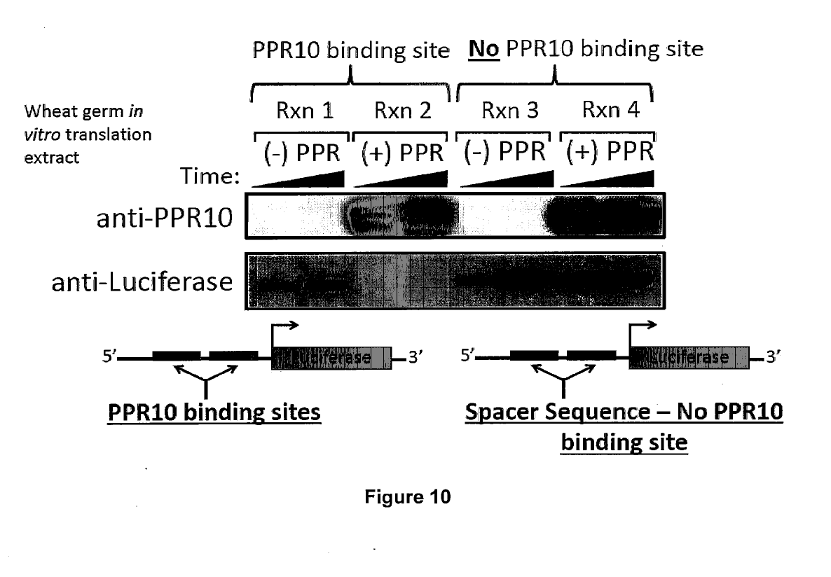

In vitro translation reactions were carried out as shown in FIG. 10. The data showed that PPR10 bound in a 5′UTR blocks translation by 80 S eukaryotic ribosomes in vitro. An in vitro transcribed mRNA encoding luciferase with the indicated 5′UTR was added to a commercial wheat germ translation extract in the presence or absence of purified recombinant PPR10.

### Gel Mobility Shift Assays

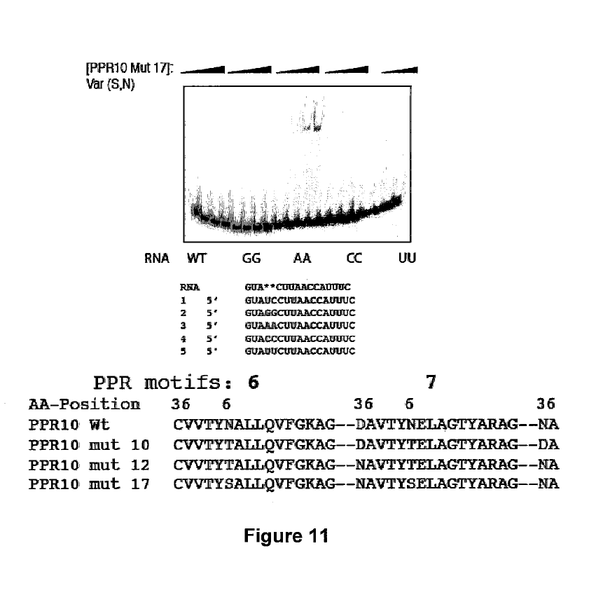

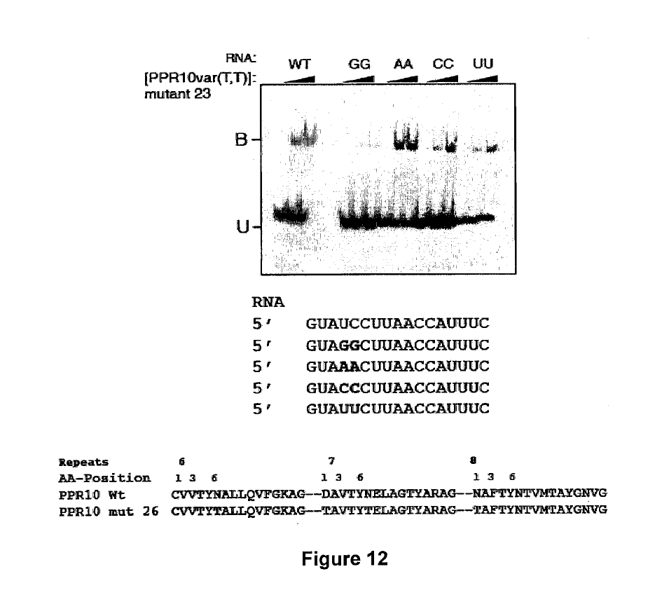

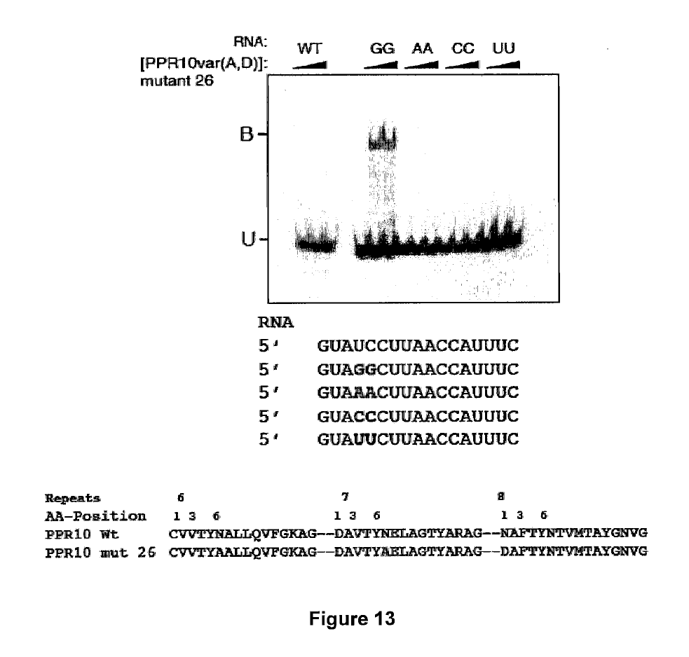

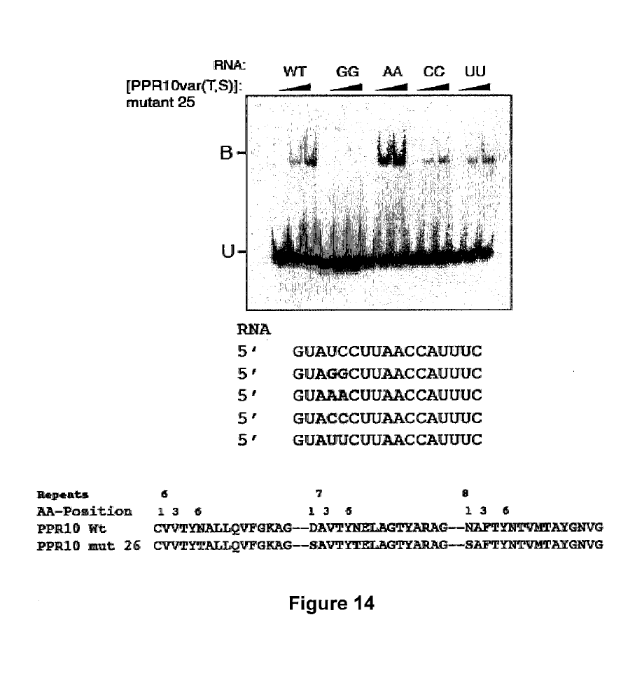

As shown in FIGS. 11 to 14, the SN variant bound to adenine with a lower affinity than the TN variant. The AD variant bound to guanine with a lower affinity than the TD variant. The TT variant and the TS variant were each found to bind to all of the RNA bases, but with the following binding preference: adenine (A)>cytosine (C), uracil (U)>guanine (G).

### EXAMPLE 3

The code as described in Examples 1 and 2 was used to score potential matches between editing sites and 188 putative RNA editing factors in order to predict which factor bound to which site in Arabidopsis chloroplasts. Five successful predictions were confirmed by analysis of plants lacking the respective editing factor (Table 1).

TABLE 1
RNA editing sites in Arabidopsis chloroplasts successfully predicted to be
bound by PPR proteins using the code of the invention described in Examples 1 and 2

| Mutant | AGI | class | Editing site | Target |
|---|---|---|---|---|
| aef1 | At3g22150 | E+ | atpF(12707) | gggagtttcggatttaataccgatattttagcaacaaatcC |
| aef2 | At1g18485 | DYW | ndhB - - - | atcctaatttttggcctaattcttcttctgatgatcgattC |
| | | | 1(97016) | |
| — | At4g37380 | DYW | ndhB - - - | gtcgttgcttttctttctgttacttcgaaagtagctgcttC |
| | | | 8(95650) | |
| aef3 | At3g14330 | DYW | psbE(64109) | gagccgacaaggcattccattaataacaggccgttttgatC |
| flv/dot4 | At4g18750 | DYW | rpoC1(21806) | cccataactaaaaaacctactttcttacgattacgaggttC |

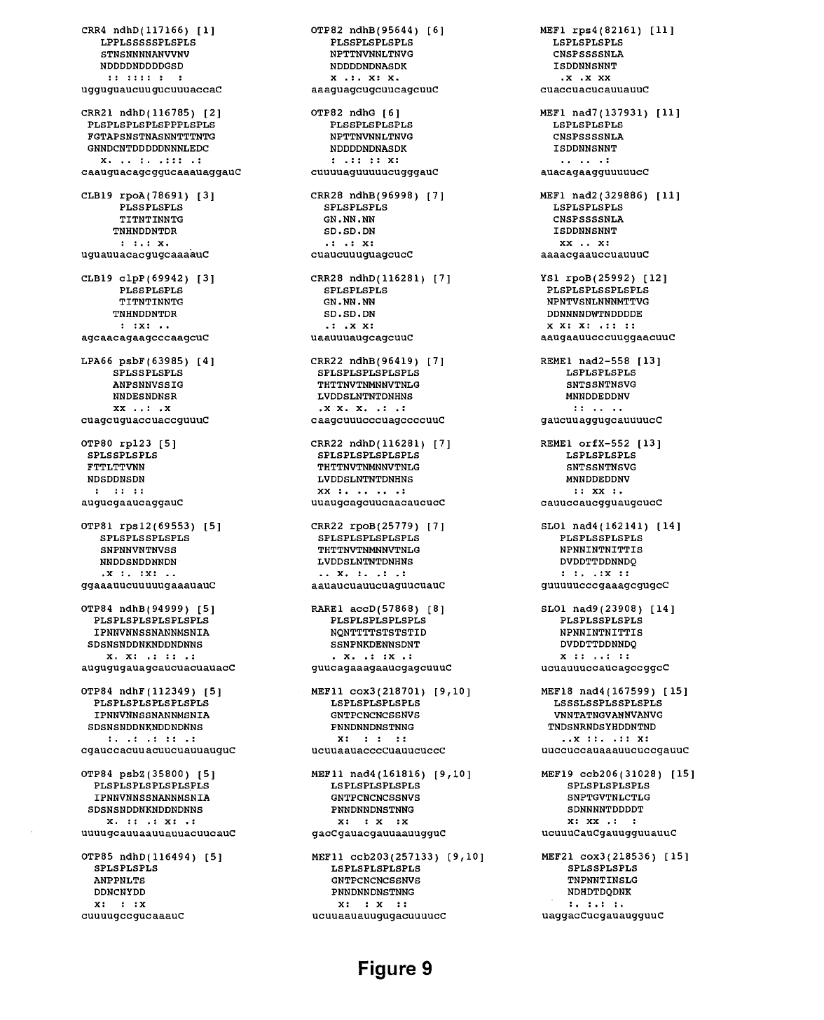

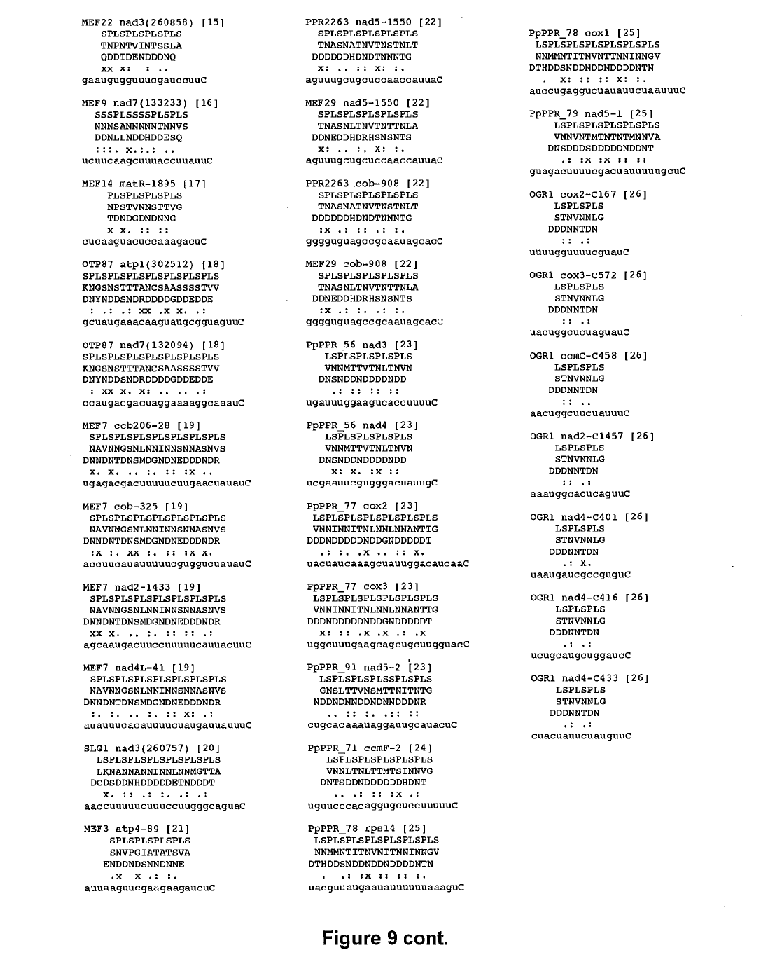

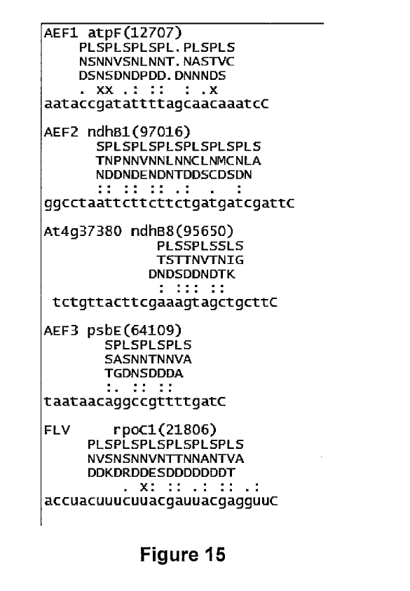

The editing factors described in Table 1 were aligned according to Examples 1 and 2, similar to that of techniques used to obtain the data of FIG. 9. The alignments of the editing factors described in Table 1 are set out in FIG. 15.

The present invention is not to be limited in scope by any of the specific embodiments described herein. These embodiments are intended for the purpose of exemplification only. Functionally equivalent products, formulations and methods are clearly within the scope of the invention as described herein.

The invention described herein may include one or more range of values (e.g. size, displacement and field strength etc). A range of values will be understood to include all values within the range, including the values defining the range, and values adjacent to the range which lead to the same or substantially the same outcome as the values immediately adjacent to that value which defines the boundary to the range.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs. The term “active agent” may mean one active agent, or may encompass two or more active agents.

Those skilled in the art will appreciate that the invention described herein is susceptible to variations and modifications other than those specifically described. The invention includes all such variation and modifications. The invention also includes all of the steps, features, formulations and compounds referred to or indicated in the specification, individually or collectively and any and all combinations or any two or more of the steps or features.

Each document, reference, patent application or patent cited in this text is expressly incorporated herein in their entirety by reference, which means that it should be read and considered by the reader as part of this text. That the document, reference, patent application or patent cited in this text is not repeated in this text is merely for reasons of conciseness.

Any manufacturer's instructions, descriptions, product specifications, and product sheets for any products mentioned herein or in any document incorporated by reference herein, are hereby incorporated herein by reference, and may be employed in the practice of the invention.

1. A recombinant polypeptide comprising at least one PPR RNA-binding domain capable of binding to a target RNA sequence, the PPR RNA-binding domain comprising at least two PPR RNA base-binding motifs comprising
a.
i. amino acid position six of a first PPR RNA base-binding motif comprises threonine (T), serine (S), or glycine (G);
ii. amino acid position one of a second adjacent PPR binding motif comprises asparagine (N), threonine (T), or serine (S); and
iii. the PPR domain is operably capable of binding to an adenine (A) RNA base in a target RNA sequence;
b.
i. amino acid position six of a first PPR RNA base-binding motif comprises threonine (T), serine (S), glycine (G), or alanine (A);
ii. amino acid position one of a second adjacent PPR binding motif comprises aspartic acid (D), threonine (T), or serine (S); and
iii. the PPR domain is operably capable of binding to a guanine (G) RNA base in a target RNA sequence;
c.
i. amino acid position six of a first PPR RNA base-binding motif comprises threonine (T) or asparagine (N);
ii. amino acid position one of a second adjacent PPR binding motif comprises asparagine (N), serine (S), aspartic acid (D), or threonine (T); and
iii. the PPR domain is operably capable of binding to a cytosine (C) RNA base in a target RNA sequence; and
d.
i. amino acid position six of a first PPR RNA base-binding motif comprises threonine (T) or asparagine (N);
ii. amino acid position one of a second adjacent PPR binding motif comprises aspartic acid (D), serine (S), asparagine (N), or threonine (T); and
iii. the PPR domain is operably capable of binding to a uracil (U) RNA base in a target RNA sequence.

2-14. (canceled)

15. The recombinant polypeptide according to claim 1, wherein each PPR RNA base-binding motif comprises between 30 and 40 amino acids.

16. The recombinant polypeptide according to claim 15, wherein the PPR RNA-binding domain comprises a plurality of pairs of PPR RNA base-binding motifs.

17. The recombinant polypeptide according to claim 16, wherein the PPR RNA-binding domain comprises a plurality of consecutively ordered pairs of PPR RNA base-binding motifs operable to bind a target RNA molecule with a target RNA sequence, each pair of PPR RNA base-binding motifs capable of specifically binding to a cytosine (C), adenine (A), guanine (G), or uracil (U) RNA base in a target RNA sequence, wherein the consecutive order of the pairs of PPR RNA base-binding motifs corresponds with the consecutive order of the target RNA sequence.

18. The recombinant polypeptide according to claim 17, wherein the target RNA molecule is RNA encoding a reporter protein selected from the group comprising his3, β-galatosidase, GFP, RFP, YFP, luciferase, β-glucuronidase, and alkaline phosphatase.

19. The recombinant polypeptide according to claim 1, wherein the target RNA molecule is RNA transcribed from chloroplast and/or mitochondrial genes.

20. The recombinant polypeptide according to claim 1, wherein the plurality of RNA base-binding motifs comprise between 2 and 40 PPR RNA base-binding motifs.

21. (canceled)

22. The recombinant polypeptide according to claim 1, wherein the PPR RNA-binding domain comprises a plurality of pairs of PPR RNA base-binding motifs operably linked via amino acid spacers.

23. The recombinant polypeptide according to claim 22, wherein the amino acid spacers are derived from SEQ ID NO: 4, or part thereof.

24. A fusion protein comprising at least one PPR RNA-binding domain according to claim 1, and an effector domain.

25. (canceled)

26. The fusion protein according to claim 24, wherein the effector domain is selected from the group comprising; Endonucleases; proteins and protein domains responsible for stimulating RNA cleavage; Exonucleases; Deadenylases; proteins and protein domains responsible for nonsense mediated RNA decay; proteins and protein domains responsible for stabilizing RNA; proteins and protein domains responsible for repressing translation; proteins and protein domains responsible for stimulating translation; proteins and protein domains responsible for polyadenylation of RNA; proteins and protein domains responsible for polyuridinylation of RNA; proteins and protein domains responsible for RNA localization; proteins and protein domains responsible for nuclear retention of RNA; proteins and protein domains responsible for nuclear export of RNA; proteins and protein domains responsible for repression of RNA splicing; proteins and protein domains responsible for stimulation of RNA splicing; proteins and protein domains responsible for reducing the efficiency of transcription; proteins and protein domains responsible for stimulating transcription; and deaminases; his3; β-galatosidase; GFP; RFP; YFP; luciferase; β-glucuronidase; and alkaline phosphatase.

27. (canceled)

28. An isolated nucleic acid encoding the recombinant polypeptide according to claim 1.

29. The isolated nucleic acid according to claim 28, having a sequence of any one of SEQ ID NOS: 5-21, or a sequence having at least 40% identity to any one of SEQ ID NOS: 5-21.

30-31. (canceled)

32. A recombinant vector comprising the nucleic acid according to claim 28.

33-36. (canceled)

37. A host cell comprising the recombinant vector of claim 32.

38-40. (canceled)

41. A composition comprising the recombinant polypeptide according to claim 1.

42. (canceled)

43. A method of regulating expression of a gene in a cell, the method comprising the step of introducing into the cell a recombinant polypeptide comprising a PPR RNA-binding domain comprising a plurality of consecutively ordered pairs of PPR RNA base-binding motifs operable to bind a target RNA molecule with a target RNA sequence, each pair of PPR RNA base-binding motifs capable of specifically binding to a cytosine (C), adenine (A), guanine (G), or uracil (U) RNA base, wherein the consecutive order of the pairs of PPR RNA base-binding motifs corresponds with the target RNA sequence; and wherein the binding of the recombinant polypeptide to the target RNA alters the expression of the gene.

44. The method according to claim 43, wherein the method is a method of activating translation, of blocking ribosome binding or ribosome scanning, of regulating RNA splicing, of stimulating RNA cleavage, or of stabilizing the transcript thereby preventing or delaying degradation.

45. A pharmaceutical composition comprising the recombinant polypeptide according to claim 1.

46-52. (canceled)

53. A kit for regulating gene expression comprising
a. a modular set of isolated nucleic acids encoding a plurality of pairs of PPR RNA base-binding motifs, the set including: at least two isolated nucleic acids each encoding a pair of PPR RNA base-binding motif capable of specifically binding to an RNA base;
b. a reagent for annealing the isolated nucleic acids of the modular set in a desired sequence to produce an isolated nucleic acid encoding a recombinant polypeptide comprising a PPR RNA-binding domain having a plurality of consecutively ordered pairs of PPR RNA base-binding motifs; and
c. optionally, a target RNA molecule with a target RNA sequence, wherein the consecutive order of the pairs of PPR RNA base-binding motifs corresponds with the target RNA sequence.

54. The kit according to claim 53, wherein each pair of PPR RNA base-binding motifs comprise between 30 and 40 amino acids.

55. The kit according to claim 53, wherein the target RNA molecule is selected from the group comprising his3, β-galatosidase, GFP, RFP, YFP, luciferase, β-glucuronidase, and alkaline phosphatase.

56. The kit according to claim 53, wherein the target RNA molecule is RNA transcribed from chloroplast and/or mitochondrial genes.

57. The kit according to claim 53, wherein the plurality of pairs of PPR RNA base-binding motifs comprise between 2 and 40 PPR RNA base-binding motifs.

58. The kit according to claim 57, wherein the plurality of pairs of PPR RNA base-binding motifs comprise 8 and 20 PPR RNA base-binding motifs.

59. The kit according to claim 53, wherein the PPR RNA-binding domain comprises a plurality of RNA base-binding motifs operably linked via amino acid spacers.

60. A method of identifying a binding target RNA sequence of a PPR RNA-binding domain comprising at least a pair of PPR RNA base-binding motifs operably capable of binding to a target RNA base, the method comprising the steps of:
a. identifying the amino acid at position six of the first PPR motif;
b. identifying the amino acid at position one of the second PPR motif; and
c. assigning to the pair of PPR motifs a binding target RNA base selected from the group comprising adenine (A), guanine (G), cytosine (C), and uracil (U);
wherein the amino acid position six of the first PPR motif is selected from the group consisting of threonine (T), serine (S), and glycine (G), amino acid position one of the second adjacent PPR binding motif is selected from the group comprising asparagine (N), threonine (T), and serine (S), and an adenine (A) RNA base is assigned to the pair of PPR motifs;
wherein the amino acid position six of the first PPR motif is selected from the group consisting of threonine (T), serine (S), glycine (G), and alanine (A), amino acid position one of the second adjacent PPR binding motif is selected from the group comprising aspartic acid (D), threonine (T), and serine (S), and a guanine (G) RNA base is assigned to the pair of PPR motifs;
wherein the amino acid position six of the first PPR motif is threonine (T) or asparagine (N), amino acid position one of the second adjacent PPR binding motif is selected from the group comprising asparagine (N), serine (S), aspartic acid (D), and threonine (T), and a cytosine (C) RNA base is assigned to the pair of PPR motifs; and
wherein the amino acid position six of the first PPR motif is threonine (T) or asparagine (N), amino acid position one of the second adjacent PPR binding motif is selected from the group comprising aspartic acid (D), serine (S), asparagine (N), and threonine (T), and a uracil (U) RNA base is assigned to the pair of PPR motifs.

61. The method according to claim 60 further comprising the step of:
d. assigning to each of a plurality of pairs of PPR motifs a binding target RNA base selected from the group comprising adenine (A), guanine (G), cytosine (C), and uracil (U);
wherein the consecutive order of the binding target RNA bases assigned corresponds with the consecutive order of the plurality of pairs of PPR RNA base-binding motifs in the PPR domain, thereby providing the target RNA sequence.

62. The method according to claim 60, wherein the binding target RNA sequence is RNA transcribed from chloroplast and/or mitochondrial genes.

63. The method according to claim 60, wherein the method identifies a plant binding target RNA sequence of a plant PPR RNA-binding domain.

64. The method according to claim 63 further comprising the step of
d. synthesizing a nucleic acid having a sequence comprising the sequence of a plurality of binding target RNA bases assigned in consecutive order to a plurality of PPR motifs.

65. (canceled)

66. An isolated nucleic acid encoding the fusion protein according to claim 24.

67. A recombinant vector comprising the nucleic acid according to claim 66.

68. A host cell comprising the recombinant vector of claim 67.


SEQUENCE LISTING (21 sequences)

SEQ ID NO: 1 - protein, 48 aa, artificial sequence
  synthetic
CVVTYTALLQVFGKAGDAVTYTELAGTYARAGDAFTYNTVMTAYGNVG

SEQ ID NO: 2 - protein, 48 aa, artificial sequence
  synthetic
CVVTYTALLQVFGKAGNAVTYTELAGTYARAGNAFTYNTVMTAYGNVG

SEQ ID NO: 3 - protein, 48 aa, Zea mays
CVVTYNALLQVFGKAGDAVTYNELAGTYARAGNAFTYNTVMTAYGNVG

SEQ ID NO: 4 - protein, 786 aa, Zea mays
MEATGRGLFPNKPTLPAGPRKRGPLLPAAPPPPSPSSLPLDSLLLHLTAPAPAPAPAPRRSHQTPTPPHSFLSPDAQVLVLAISSHPLPTLAAFLASRRDELLRADITSLLKALELSGHWEWALALLRWAGKEGAADASALEMVVRALGREGQHDAVCALLDETPLPPGSRLDVRAYTTVLHALSRAGRYERALELFAELRRQGVAPTLVTYNVVLDVYGRMGRSWPRIVALLDEMRAAGVEPDGFTASTVIAACCRDGLVDEAVAFFEDLKARGHAPCVVTYNALLQVFGKAGNYTEALRVLGEMEQNGCQPDAVTYNELAGTYARAGFFEEAARCLDTMASKGLLPNAFTYNTVMTAYGNVGKVDEALALFDQMKKTGFVPNVNTYNLVLGMLGKKSRFTVMLEMLGEMSRSGCTPNRVTWNTMLAVCGKRGMEDYVTRVLEGMRSCGVELSRDTYNTLIAAYGRCGSRTNAFKMYNEMTSAGFTPCITTYNALLNVLSRQGDWSTAQSIVSKMRTKGFKPNEQSYSLLLQCYAKGGNVAGIAAIENEVYGSGAVFPSWVILRTLVIANFKCRRLDGMETAFQEVKARGYNPDLVIFNSMLSIYAKNGMYSKATEVFDSIKRSGLSPDLITYNSLMDMYAKCSESWEAEKILNQLKCSQTMKPDVVSYNTVINGFCKQGLVKEAQRVLSEMVADGMAPCAVTYHTLVGGYSSLEMFSEAREVIGYMVQHGLKPMELTYRRVVESYCRAKRFEEARGFLSEVSETDLDFDKKALEAYIEDAQFGR

SEQ ID NO: 5 - DNA, 24 bp, artificial sequence
  synthetic
ggtctgttgccagacgcattcacg

SEQ ID NO: 6 - DNA, 24 bp, artificial sequence
  synthetic
cgtgaatgcgtctggcaacagacc

SEQ ID NO: 7 - DNA, 30 bp, artificial sequence
  synthetic
gctgtgacgtacaccgagctcgccggaacg

SEQ ID NO: 8 - DNA, 30 bp, artificial sequence
  synthetic
cgttccggcgagctcggtgtacgtcacagc

SEQ ID NO: 9 - DNA, 34 bp, artificial sequence
  synthetic
cacctggagcaacgcggtgtacgtgacgacgcac

SEQ ID NO: 10 - DNA, 25 bp, artificial sequence
  synthetic
cgtgaatgcgtttggcaacagaccc

SEQ ID NO: 11 - DNA, 25 bp, artificial sequence
  synthetic
gggtctgttgccaaacgcattcacg

SEQ ID NO: 12 - DNA, 31 bp, artificial sequence
  synthetic
gaacggctgccagccaaacgctgtgacgtac

SEQ ID NO: 13 - DNA, 32 bp, artificial sequence
  synthetic
cggtgtacgtcacagcgtttggctggcagccg

SEQ ID NO: 14 - DNA, 34 bp, artificial sequence
  synthetic
ggagcagaacggctgccagccaaacgctgtgacg

SEQ ID NO: 15 - DNA, 34 bp, artificial sequence
  synthetic
cgtcacagcgtttggctggcagccgttctgctcc

SEQ ID NO: 16 - DNA, 24 bp, artificial sequence
  synthetic
ggtctgttgccagacgcattcacg

SEQ ID NO: 17 - DNA, 24 bp, artificial sequence
  synthetic
cgtgaatgcgtctggcaacagacc

SEQ ID NO: 18 - DNA, 23 bp, artificial sequence
  synthetic
gctgccagccaagcgctgtgacg

SEQ ID NO: 19 - DNA, 23 bp, artificial sequence
  synthetic
cgtcacagcgcttggctggcagc

SEQ ID NO: 20 - DNA, 32 bp, artificial sequence
  synthetic
gtctgttgccaagcgcattcacgtacaacacc

SEQ ID NO: 21 - DNA, 32 bp, artificial sequence
  synthetic
ggtgttgtacgtgaatgcgcttggcaacagac